(19) United States
(12) Patent Application Publication
(10) Pub. No.: US 2022/0177599 A1
(43) Pub. Date: Jun. 9, 2022

(54) DUAL CHIMERIC ANTIGEN RECEPTOR TARGETING EPCAM AND ICAM-1

(71) Applicant: Affyimmune Therapeutics, Inc., Natick, MA (US)

(72) Inventor: Moonsoo JIN, New York, NY (US)

(21) Appl. No.: 17/457,785

(22) Filed: Dec. 6, 2021

Related U.S. Application Data
(60) Provisional application No. 63/123,071, filed on Dec. 9, 2020.

(51) Int. Cl. C07K 16/30; C07K 14/705; C07K 14/725; C07K 14/72; A61K 35/17; A61P 35/00

(52) U.S. Cl. CPC C07K 16/30; C07K 14/70546; C07K 14/7051; C07K 2317/622; A61K 35/17; A61P 35/00; C07K 2319/03; C07K 14/72

(57) ABSTRACT

The present invention relates to low affinity dual chimeric antigen receptors (CARs), which provide cytotoxicity against heterogenous tumors and alleviate on-target, off-tumor toxicities. The dual CARs are constructed to have two binding domains bearing reduced affinities of 50 nM to 50 μM, one of which is the inserted or I domain of the α_{L} subunit of Lymphocyte function-associated antigen-1, and the other one is scFv of EpCAM antibody. The dual CAR T cells of the present invention provide enhanced anti-tumor activity and reduced rate of tumor relapse.

This application claims priority to U.S. Provisional Application No. 63/123,071, filed Dec. 9, 2020; the contents of which is incorporated herein by reference in its entirety.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Nov. 30, 2021 and a size of 20.4 kilobytes. The Sequence Listing filed via EFS-Web is part of the specification and is hereby incorporated in its entirety by reference herein.

### FIELD OF THE INVENTION

The present invention relates to a bispecific dual chimeric antigen receptor (CAR) simultaneously targeting both EpCAM and inducible ICAM-1. The dual CAR comprises EpCAM single-chain variable fragment and the inserted or I domain of the α_{L} subunit of Lymphocyte function-associated antigen (LFA)-1. The dual CAR has functionally sufficient but low affinity to both EpCAM and ICAM-1, which provides cytotoxicity against heterogenous tumors and mitigates cytotoxicity to normal tissues.

### BACKGROUND OF THE INVENTION

Immunotherapy is emerging as a highly promising approach for the treatment of cancer. Genetically modifying T cells with CARs is a common approach to design tumor-specific T cells. CAR (chimeric antigen receptor)-T cells targeting tumor-associated antigens can be infused into patients (adoptive cell transfer or ACT) representing an efficient immunotherapy approach. The advantage of CAR-T technology compared with chemotherapy or antibody is that reprogrammed engineered T cells can proliferate and persist in the patient and work like a living drug.

CAR T cell therapy is a rapidly emerging immunotherapy approach which reprograms T cell specificity and function using a synthetic antigen receptor^{1,2}. Adoptive transfer of CAR T cells has produced remarkable responses across a range of B-cell leukemias and lymphomas in which all other treatment options have been exhuasted³⁻⁵. Early clinical trial results also indicate encouraging clinical efficacy of CAR T cell therapy against relapsed or refractory multiple myeloma^{6,7}. In spite of high rates of initial response, however, relapses involving diminished or complete loss of cell-surface antigen expression are observed in approximately 30-50% of patients who achieve remission after treatment with anti-CD19 CAR T cells, usually within one year of treatment⁸⁻¹⁰. Relapses associated with antigen loss have also been reported with CARs directed against other targets, such as CD22 and B-cell maturation antigen, underscoring antigen escape as a significant and common impediment to the success of CAR T cell therapy^{6, 7, 11}. Going beyond hematological cancers, antigen escape is likely to be an even greater challenge in solid tumors, which are generally composed of cells with heterogeneous antigen expression¹²⁻¹⁴.

CAR molecules are composed of synthetic binding moieties, typically an antibody-derived single chain fragment variable (svFv) or any native antigen-sensing element, fused to intracellular signaling domains composed of the TCR zeta chain and costimulatory molecules such as CD28 and/or 4-1BB. The advantages of CAR mediated targeting include: 1) the provision of activation, proliferation, and survival signals in-cis via a single binding event, compared to the natural, non-integrated TCR and costimulatory signaling; 2) the ability to bypass the downregulation of MHC by tumor cells through MHC-independent antigen recognition; and 3) a reduced activation threshold as well as recognition of tumor cells with low antigen density enabled by the high affinity interaction between CAR and antigen.

The ideal CAR target antigen would be a native, surface-exposed tumor neoantigen that is highly expressed and is undetectable in healthy tissues. However, due to the implicit rarity of such antigens, many commonly targeted solid tumor antigens, are also expressed by non-tumor tissues, albeit at lower levels. CAR molecules with high affinity to such antigens can lead to collateral targeting of healthy tissues resulting in on-target, off-tumor toxicity, a major limiting factor to the progress of CAR T cell therapy to date.

EpCAM (Epithelial Cell Adhesion Molecule) (CD326) antigen is a 35 kDa cell surface glycoprotein that is encoded by EpCAM gene. EpCAM plays a crucial role in cell adhesion, growth, proliferation, inflammation, cancer and metastasis. EpCAM is highly overexpressed in many types of tumors such as breast cancer, ovarian cancer, non-small cell lung cancer, pancreas cancer, stomach cancer, colon cancer and colorectal cancer. EpCAM is also expressed in many normal tissues but its expression in tumor tissues is significantly higher.

High affinity EpCAM CAR-T cells recognize epithelial cell adhesion molecule-expressing cells: both normal epithelial tissues with low levels of EpCAM, and carcinomas expressing it at considerably higher levels. The recognition of antigen both on normal, non-target cells as well as on cancer cells can lead to both unwanted toxicity and T cell exhaustion.

Intercellular adhesion molecule-1 (ICAM-1), GenBank Accession Nos. NM_000201, NP_000192, is the ligand for α_{L}β₂ integrin, and its N-terminal domain (D1) binds to the α_{L} I domain through the coordination of ICAM-1 residue Glu-34 to the MIDAS metal. ICAM1 is typically expressed on endothelial cells and cells of the immune system. ICAM-1 binds to integrins of type α_{L}β₂ and α_{M}β₂. ICAM-1 is upregulated in several carcinomas and the associated stroma²⁴ as well as in inflammatory conditions. Aside from diseased tissues, ICAM-1 is basally expressed in several cell types including endothelial cells, immune cells, and some epithelial cells. ICAM-1 is a biomarker prevalent in various types of tumors; it can be upregulated in response to inflammatory mediators, including IL-1, IFN-γ and TNF-α, and subsequently facilitates leukocyte adhesion and transmigration by binding to lymphocyte function-associated antigen-1 (LFA-1)²¹⁻²⁴.

Adoptive transfer of chimeric antigen receptor (CAR) T cells has demonstrated unparalleled responses in hematological cancers, yet antigen escape and tumor relapse occur frequently. Due to heterogeneous tumor antigen expression, difficulty grows in the treatment of patients with solid tumors. Moreover, severe toxicity associated with on-target off-tumor targeting poses an additional challenge to effective CAR T cell therapy in solid tumors.

There exists a need for CARs with improved therapeutic index, i.e., CARs that can kill tumor while minimizing systemic toxicity.

### BRIEF DESCRIPTION OF THE DRAWINGS

FIG. 1. EpCAM CAR, ICAM-1 CAR, Tandem CAR, and bicistronic CAR Schematic representation of the lentiviral vector encoding EpCAM CAR, ICAM-CAR, and two dual CARs. The expression of CAR constructs is driven by the elongation factor 1α (EF1α) promoter, and co-expression of human SSTR2 is under the control of a ribosome-skipping sequence P2A. A c-Myc tag is introduced at the N-terminus for CAR detection. SS, signal sequence; CD8 Hing-TM, CD8 hinge and transmembrane domains; CD28 Cyt, CD28 cytosolic domain. 4-1BB Cyt, 4-1BB cytosolic domain.

EpCAM CAR: The antigen binding domain contains scFv derived from UBS54 monoclonal antibodies.

ICAM-1 CAR: The antigen binding domain contains I domain with F292A mutation.

Tandem CAR: One CAR containing two antigen binding domains. (G₄S)₂ is a linker of 4 glycines and one serine repeating twice.

Bicistronic CAR: Two separate CARs expressed in a same cell.

FIG. 2. Dual CAR attacking tumor cells.

Schematic representation of bicistronic dual CAR and tandem CAR attacking tumor cells.

FIG. 3. Affinity determination of EpCAM CARs.

The affinity of C215 and UBS54 CARs was determined by staining CAR-expressing Jurkat T cells with serially diluted AF647-conjugated EpCAM. K_{D} values were calculated using the one-site nonlinear regression model. Data represent mean±s.d. (n=3). MFI: mean fluorescent intensity.

FIGS. 4A and 4B (4B-1 and 4B-2). Characterization of EpCAM CAR T cells in vitro and tumor cell lines.

A. Cytolytic activity of CAR T cells against EpCAM-expressing target cells and negative control U-251 cells. CAR T cells were co-incubated with target cells at an E:T ratio of 1:1, and 24 hours later the percentage of target cell viability was normalized to luminescence from target cells without T cell treatment. Data represent mean±s.d. (n=3). Statistical comparison between C215 and UBS54 CAR T cells was performed by unpaired, two-tailed t-test (ns, not significant; *, P<0.05; **, P<0.01; ***, P<0.001). In each target cell, from left to right: NT, C215CAR, and UBS54CAR

B-1 and B-2. Cytokine levels measured in culture supernatants from the co-culture of CAR T cells and target cells described in b (n=2). In each target cell, from left to right: NT, C215CAR, and UBS54CAR

FIGS. 5A-5M. Lower-affinity EpCAM CAR T cells mediate complete remission in gastric and pancreatic cancer models.

A. Schematic of the intraperitoneal SNU-638 tumor model. NSG mice were intraperitoneally implanted with 0.5×10⁶ SNU-638 cells. 7 days later, mice were either left untreated or treated with NT, C215, or UBS54 CAR T cells (10×10⁶ cells/mouse) via intraperitoneal injection.

B. Representative bioluminescence images of SNU-638 engrafted NSG mice.

C. Quantitation of total body bioluminescence intensity from two independent experiments. Data represent mean s.d. (n=6-7). Two-way ANOVA with Tukey's multiple comparisons test, ns, not significant; *, P<0.05; ****, P<0.0001.

D. Kaplan-Meier survival curves. Log-rank (Mantel-Cox) test, ns, not significant; **, P<0.01; ***,P<0.001.

E. Schematic of the systemic MKN-45 tumor model. 5 days after intravenous (i.v.) inoculation of 0.5×10⁶ MKN-45 cells, mice were treated with T cells (10×10⁶ cells/mouse, i.v.) or left untreated.

F. Whole-body bioluminescence images of MKN-45 engrafted NSG mice.

G. Quantitation of total body bioluminescence intensity. Data represent mean±s.d. (n=4).

H. Kaplan-Meier survival curves.

I. Summary of body weight changes over time (n=4).

J. PET/CT images showing CAR T cell biodistribution following T cell infusion.

K. Schematic of the orthotopic Capan-2 tumor model. 0.1×10⁶ Capan-2 cells were implanted orthotopically into the pancreas, and after 15 days, mice received 10×10⁶ NT or UBS54 CAR T cells intravenously.

L. Whole-body bioluminescence images of Capan-2 engrafted NSG mice.

M. Quantitation of total body bioluminescence intensity. Data represent mean±s.d. (n=2-3).

FIG. 6. Lower affinity EpCAM CAR T cells control tumor growth in gastric cancer patient derived xenograft models.

A. Tumor volumes in NSG mice without treatment (No T), or treated with NT, or lower-affinity UBS54 EpCAM CAR T cells (n=3 for each PDX model).

B. Kaplan-Meier survival analysis of three independent PDX experiments described in c. Statistical significance was determined using log-rank (Mantel-Cox) test, ns, not significant; ****, P<0.0001.

C. Cytokine levels measured in mouse plasma after T cell infusion. Data are pooled from independent PDX44 and PDX55 experiments and are shown as individual values (n=4 mice). HLOQ=higher limit of quantification.

f PET/CT images showing CAR T cell accumulation at tumor sites. CAR T cells contracted when tumors were eradicated on day 14 following T cell infusion.

FIGS. 7A-7D. Simultaneous targeting of EpCAM and ICAM-1 facilitates cytotoxicity against heterogenous tumors in vitro.

A. Representative flow cytometry plots showing CAR expression and CD4/CD8 phenotype following transduction of human primary T cell. The production of dual CAR T cells was performed independently at least four times. The percentage of CAR positive, CD3, and CD4/CD8 subsets are shown (n=4).

B. Bioluminescence-based cytotoxicity assay measuring cytolytic activity of F292A, UBS54, and dual CAR T cells. A heterogeneous population (100%, 50%, or 3% EpCAM⁺) of SNU-638 or MKN-45 tumor cells were co-incubated with T cells at an E:T ratio of 1:1 for 48 hours. The percentage of target cell viability was normalized to luminescence from No T cohort. Data represent mean±s.d. from quadruplicates, unpaired, two-tailed t-test, *, P<0.05; **, P<0.01; ***, P<0.001, ****, P<0.0001.

C. The upregulation of CD137 expression on T cells was measured after stimulation by SNU-638 or MKN-45 tumor cells (50% EpCAM⁺) for 24 hours (mean±s.d., n=4).

D. Cytokine production by NT, F292A, UBS54, and dual CAR T cells upon co-culture with SNU-638 or MKN-45 tumor cells (50% EpCAM⁺) for 24 hours (n=2).

FIGS. 8A-8H. EpCAM-ICAM-1 dual CAR T reduces tumor recurrence rate in a subcutaneous gastric cancer model.

A. Schematic of the subcutaneous SNU-638 tumor model. NSG mice were subcutaneously implanted with 1×10⁶ SNU-638 cells and treated 7 days later with UBS54 or dual CAR T cells (10×10⁶cells/mouse) via tail vein injection.

B. Representative bioluminescence images of SNU-638-engrafted NSG mice.

C. Quantitation of total-body bioluminescence intensity from independent experiments using three batches of donor-matched CAR T cells. Data are shown as mean±SD (n=10-20). Statistical significance determined by two-way ANOVA with Tukey multiple comparisons test.

D. Tumor volumes for mice in the No T (n=11), UBS54 (n=20), and dual (n=10) cohorts.

E. Incidence of tumor relapse (total-body bioluminescence intensity >2×10⁸ photons/second, n=10-20). P values determined by log-rank (Mantel-Cox) test.

F. EpCAM cell-surface densities in relapsed tumors of mice sacrificed 8 to 12 weeks after UBS54 CAR T-cell treatment (mean±SD, n=2-3). Fold change in MFI of EpCAM was normalized to unstained cells.

G. Serum IFNg and perforin were measured weekly during the first 3 weeks following T-cell administration.

H. Longitudinal PET/CT imaging to evaluate CAR T cell expansion in vivo (n=2/cohort). Subcutaneous tumors are indicated by white arrowheads.

FIGS. 9A-9G. EpCAM-ICAM-1 dual CAR T cells show enhanced anti-tumor function in a heterogeneous gastric cancer MKN-45 model.

NSG mice were subcutaneously implanted with a heterogeneous population of MKN-45 cells (90% wild-type, 10% EpCAM-negative, 1×10⁶ cells/mouse) and 5 days later received F292A, UBS54 or dual CAR T cells CAR T cells (10×10⁶ cells/mouse) via tail vein injection.

A. Bioluminescence images showing mixed tumor growth over time (n=3-4).

B. Total body bioluminescence intensity shown as average values (mean±s.d.).

C. Tumor volume measurements over time (mean±s.d., two-way ANOVA with Tukey's multiple comparisons test, ns, not significant; *, P<0.05).

D. Survival analysis using log-rank (Mantel-Cox) test, ns, not significant; *, P<0.05, n=3-4.

E. Changes in EpCAM and ICAM-1 cell-surface expression in tumor cells following CAR T cell treatment (n=2-4).

F. Longitudinal PET/CT images showing ¹⁸F-NOTAOCT uptake in CAR T cells. Subcutaneous tumors are indicated by white arrow heads.

G. Serum IFN-γ and perforin levels measured at 2, 9, and 16 days post T cell administration. On day 2, serum IFN-γ in the UBS54-treated mouse was above the higher limit of quantification (HLOQ).

FIGS. 10A-10D. EpCAM-ICAM-1 dual CAR T mediates longer lasting remission in a heterogeneous SNU-638 tumor model.

A. Bioluminescence images showing mixed tumor growth after receiving no treatment (No T; n=3) or treatment with UBS54 (n=5) or dual (n=6) CAR T cells.

B. Tumor growth shown as average values of total-body BLI or tumor volume, respectively (mean±SD, n=3-6). P values determined by two-way ANOVA with Tukey multiple comparisons test.

C. Percentage of mice bearing tumors <500 mm³ (n=3-6). P values determined by log-rank (Mantel-Cox) test.

D. Changes in EpCAM and ICAM-1 cell-surface expression in tumor cells 5 to 10 weeks following CAR T-cell treatment (mean±SD, n=2-3).

FIG. 11. The response (tumor size and luminescence) to different treatments (ICAM-1 CAR, EpCAM CAR, tandem dual CAR, and bicistronic dual CAR, from top to bottom in the figure) on MKN-45 (90% EpCAM positive, ICAM-1 low) tumor cells implanted in mice.

FIG. 12. The amino acid sequence of VH of UBS-54 (SEQ ID NO: 14).

### DETAILED DESCRIPTION OF THE INVENTION

### Definitions

As used herein, “about” refers to ±10% of the recited value.

As used herein, “adoptive T cell therapy” involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells which can attack and kill cancer.

As used herein, “affinity” is the strength of binding of a single molecule (e.g., I domain, or EpCAM antibody) to its ligand (e.g., ICAM-1, or EpCAM). Affinity is typically measured and reported by the equilibrium dissociation constant (K_{D} or Kd), which is used to evaluate and rank order strengths of bimolecular interactions.

As used herein, a “chimeric antigen receptor (CAR)” is a receptor protein that has been engineered to give T cells the new ability to target a specific protein. The receptor is chimeric because they combine both antigen-binding and T-cell activating functions into a single receptor. CAR is a fused protein comprising an extracellular domain capable of binding to an antigen, a transmembrane domain, and at least one intracellular domain. The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, “integrin” or “integrin receptor” (used interchangeably) refers to any of the many cell surface receptor proteins, also referred to as adhesion receptors which bind to extracellular matrix ligands or other cell adhesion protein ligands thereby mediating cell-cell and cell-matrix adhesion processes. Binding affinity of the integrins to their ligands is regulated by conformational changes in the integrin. Integrins are involved in physiological processes such as, for example, embryogenesis, hemostasis, wound healing, immune response and formation/maintenance of tissue architecture. Integrin subfamilies contain a beta-subunit combined with different alpha-subunits to form adhesion protein receptors with different specificities.

“Lymphocyte function-associated antigen-1”, “LFA-1”, “α_{L}β₂ integrin” or “CD18/CD11a” refers to a member of the leukocyte integrin subfamily. LFA-1 is found on all T-cells and also on B-cells, macrophages, neutrophils and NK cells and is involved in recruitment to the site of infection. It binds to ICAM-1 on antigen-presenting cells and functions as an adhesion molecule.

As used herein, “I domain” refers to the inserted or I domain of the α_{L} subunit of LFA-1, and is an allosteric mediator of ligand binding to LFA-1. I domain is a native ligand of ICAM-1. The ligand binding site of the I domain, known as a metal ion-dependent adhesion site (MIDAS), exists as two distinct conformations allosterically regulated by the C-terminal α7 helix. A wild-type (WT) I domain encompasses amino acid residues 130-310 of the 1145 amino acid long mature α_{L} integrin subunit protein (SEQ ID NO: 1, which is the amino acid residues 26-1170 of GenBank Accession No. NP_002200). All numbering of amino acid residues as used herein refers to the amino acid sequence of the mature α_{L} integrin (SEQ ID NO: 1), wherein residue 1 of SEQ ID NO: 1 corresponds to residue 26 of the sequence of GenBank Accession No. NP_002200. SEQ ID NO: 1 is published as SEQ ID NO: 1 in U.S. Pat. No. 10,428,136, which is incorporated herein by reference.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for engineering an scFv are known to a person skilled in the art.

“Somatostatin receptor type 2 (SSTR2)” is a receptor for somatostatin-14 and -28. Somatostatin acts at many sites to inhibit the release of many hormones and other secretory proteins. The biologic effects of somatostatin are probably mediated by a family of G protein-coupled receptors that are expressed in a tissue-specific manner. SSTR2 is a member of the superfamily of receptors having seven transmembrane segments and is expressed in highest levels in cerebrum and kidney. A full molecule of human SSTR2 has 369 amino acids and its sequence is shown as GenBank Accession No. NP_001041. A “truncated SSTR2”, as used herein, refers to a C-terminus shortened human SSTR2, which contains 1-314 amino acid residues of human SSTR2 with a deletion of the C-terminus beyond amino acid 314.

As used herein, a “tumor antigen” means a biological molecule having antigenicity, expression of which causes cancer.

### DESCRIPTION

EpCAM is a surface antigen that has been found to be frequently upregulated in a wide variety of carcinomas, including colorectal, gastric, pancreatic, and endometrial cancers. Single EpCAM CAR T cells are often unable to completely eliminate tumors with heterogeneous expression of EpCAM, resulting in outgrowth of EpCAM low or negative tumors.

The present invention provides dual CAR T cells, which target EpCAM and ICAM-1 simultaneously and are more resistant to antigen escape. The dual CARs complement EpCAM CAR with additional targeting of ICAM-1, owing to the inducible nature of ICAM-1 by inflammatory T-cell cytokines. The dual CARs improve the efficacy of CAR T cells against the EpCAM-overexpressing tumors and prevent the immune evasion of antigen-negative variants.

Dual CAR T cells that additionally target ICAM-1 can eradicate tumors with heterogenous expression of EpCAM, irrespective of initial ICAM-1 expression, and is less susceptible to tumor relapse. Due to the nature of ICAM-1 being inducible by proinflammatory cytokines, addition of CAR against ICAM-1 complements and boosts the activity of CARs against EpCAM. To improve the safety, dual CARs with lower affinities to EpCAM and ICAM-1 (Kd higher than 50 nM or 100 nM) are selected. Higher affinity does not necessarily improve the efficacy but instead it could raise the risk of off-tumor or autoimmune responses. CARs derived from scFvs generally have higher affinity (K_{d}: 1-100 nM) compared to TCRs, usually resulting in severe toxicities owing to off-tumor recognition.

The present invention is directed to a dual CAR that has greater resistance to antigen escape by simultaneously targeting EpCAM and ICAM-1. The present invention provides dual CARs targeting EpCAM and ICAM-1, which have broad anti-tumor applicability. Dual CAR or bispecific CAR are used interchangeably in this application, referring CARs targeting both EpCAM and ICAM-1.

The dual CAR of the present invention comprises: (a) a single-chain variable fragment (scFv) against EpCAM, (b) a human I domain of the α_{L} subunit of human lymphocyte function-associated antigen-1 (I domain), (c) at least one transmembrane domain, (d) at least one co-stimulatory domains, and (iv) at least one activating domain. In one embodiment, the dual CAR is bicistronic. In another embodiment, the dual CAR is tandem. The bispecific CAR optionally comprises a reporter molecule such as SSTR2.

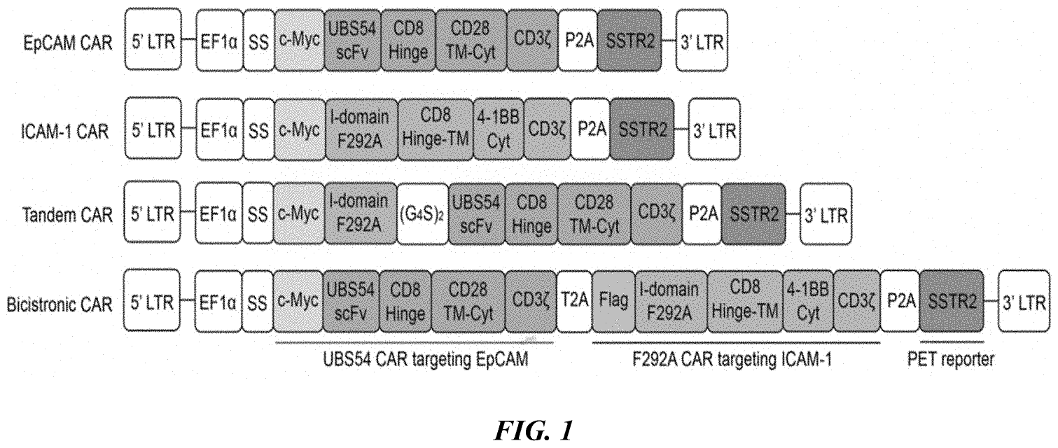

FIG. 1 is a schematic representation of the lentiviral vector encoding EpCAM CAR, ICAM-CAR, and two dual CARs (tandem dual CAR, and bicistronic dual CAR). FIG. 1 shows one embodiment of the invention, however, the present invention is not limited to the embodiment drawn in FIG. 1.

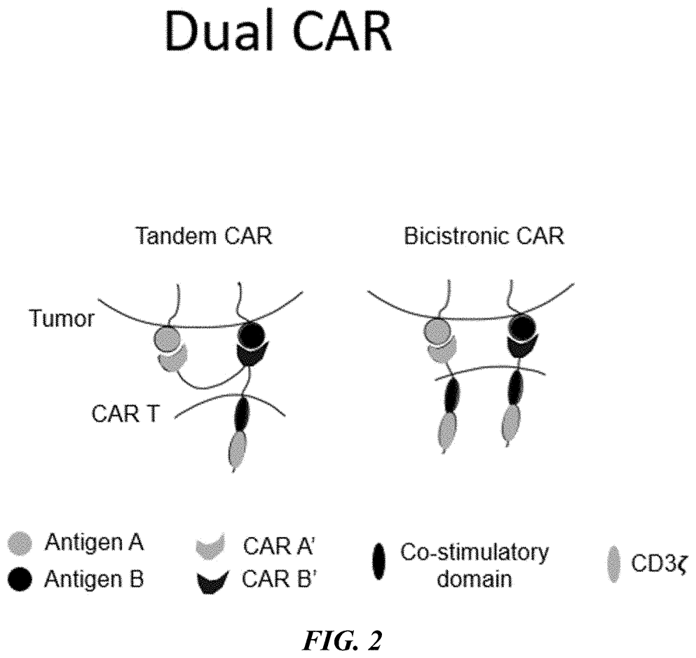

FIG. 2 is a schematic representation of bicistronic dual CAR and tandem CAR attacking tumor cells.

In one embodiment, the dual CAR is bicistronic CAR. The CAR comprises an EpCAM CAR targeting EpCAM and an ICAM-1 CAR targeting ICAM-1, wherein the EpCAM CAR comprises scFv against EpCAM, one transmembrane domain, one or more co-stimulatory domains, and one activating domain, and the ICAM-1 CAR comprises an I domain, one transmembrane domain, one or more co-stimulatory domains, and one activating domain. The transmembrane domain, the co-stimulatory domain(s), and the activating domain of the EpCAM CAR and the ICAM-1 CAR can be the same or different. The EpCAM CAR can be N-terminal or C-terminal to the ICAM-1 CAR. Each CAR optionally comprises a tag (e.g., a Myc tag or a FLAG tag) at the N-terminus or C-terminus for CAR detection. In this embodiment, the dual CAR incorporates two CARs each independently encoding CD28 or 41BB costimulatory domain. The greater cytotoxic activity and cytokine secretion of dual CAR T are likely from complementary and additive costimulatory signals through both CD28 and 41BB when CAR is engaged with two antigens.

In another embodiment, the dual CAR is tandem. The tandem CAR comprises from N-terminus to C-terminus scFv against EpCAM, I domain, a transmembrane domain, one or more co-stimulatory domains, and an activating domain. In this embodiment, the bispecific CAR employs the same costimulatory domain(s) and the same activating domain for both EpCAM and ICAM-1 antigens. The bispecific CAR optionally comprises a tag (e.g., a Myc tag or a FLAG tag) at the N-terminus or C-terminus for CAR detection.

In yet another embodiment, the bispecific tandem CAR comprises from N-terminus to C-terminus I domain, scFv against EpCAM, a transmembrane domain, one or more co-stimulatory domains, and an activating domain. In this embodiment, the bispecific CAR employs the same costimulatory domain(s) and the same activating domain for both EpCAM and ICAM-1 antigens. The bispecific CAR optionally comprises a tag (e.g., a Myc tag or a FLAG tag) at the N-terminus or C-terminus for CAR detection.

CAR T cells with target affinities in the 50 nM to 50 μM range can avoid targeting healthy tissue with basal antigen expression while simultaneously exhibiting comparable potency and long-term efficacy against tumor tissue with high target expression. The 50 nM to 50 μM affinity CAR enables T cells to neglect normal tissues having low EpCAM expression. High affinity and avidity interactions by low nanomolar affinity EpCAM-CAR can reduce T cells' propensity for serial killing, potentially causing exhaustion or increased susceptibility to activation-induced cell death.

CAR T cells comprising the bispecific CARs of the present invention preferably have sufficient affinities targeting both EpCAM and ICAM-1, but do not have such a high affinity that would attack normal cells. CAR T cells comprising the bispecific CARs of the present invention have improved efficacy and safety over conventional CARs, as they are capable of lysing cells overexpressing one of the two target antigens, while sparing normal cells with much lower densities.

In one embodiment, the bispecific CAR binds to EpCAM with an affinity between about 50 nM and about 50 μM, preferably between about 80 nM and about 20 μM, or between about 100 nM and about 10 μM.

In one embodiment, the bispecific CAR binds to ICAM-1 with an affinity between about 50 nM and about 20 μM, preferably between about 80 nM and about 25 μM, or between about 100 nM and about 20 μM.

In one embodiment, the bispecific CAR binds to EpCAM with an affinity between about 50 nM and about 50 μM, preferably between about 80 nM and about 20 μM, or between about 100 nM and about 10 μM, and binds to ICAM-1 with an affinity between about 50 nM and about 20 μM, preferably between about 80 nM and about 25 μM, or between about 100 nM and about 20 μM.

Huls, et al (Nat Biotechnol. 17, 276-281 (1999)) isolated a human monoclonal antibody UBS-54 (UBS-54) that was specific for EpCAM. The VH and VL sequences of UBS-54 are shown in U.S. Pat. No. 7,777,010, and are incorporated herein by reference. The inventors have prepared EpCAM-CAR with scFv of UBS-54 and found the CAR affinity to be about 250 nM. UBS-54 is suitable for the dual CAR of the present invention.

The amino acid sequence of VH of UBS-54 (SEQ ID NO: 14) is shown in FIG. 12; the CDR-H3 of USB-54 has the amino acid sequence of DPFLHY (SEQ ID NO: 2). The inventor also used several low scFv's with similar or lower CAR affinity than that of UBS-54, each having the same VH and VL sequences as those of UBS-54, except CDR-H3 having one amino acid variation from that of UBS-54.

In one embodiment, the dual CAR of the present invention comprises EPCAM scFv, wherein the CDR-H3 has the amino acid sequence DPFLHY (SEQ ID NO: 2), DPFLHA (SEQ ID NO: 3), DPFLHL (SEQ ID NO: 4), DPFLHV (SEQ ID NO: 5), DPFLHF (SEQ ID NO: 6), APFLHY (SEQ ID NO: 7), or DPFAHY (SEQ ID NO: 8). CAR having these CDR-H3's have lower affinity than or comparable to CAR having scFv of UBS-54.

The scFv may further comprise heavy chain variable CDR1 of the amino acid sequence of GGTFSSY (SEQ ID NO: 9) and heavy chain variable CDR2 of the amino acid sequence of IPIFGT (SEQ ID NO: 10). The scFv may further comprise light chain variable CDR1 of the amino acid sequence of RSSQSLLHSNGYNYLD (SEQ ID NO: 11), the light chain variable CDR2 of the amino acid sequence of LGSNRAS (SEQ ID NO: 12), and the light chain variable CDR3 of the amino acid sequence of MQALQTFT (SEQ ID NO: 13). The above low affinity EPCAM scFv are suitable for the dual CAR of the present invention.

In one embodiment, the EPCAM scFv of the dual CAR comprises the same VH as those of UBS-54 (SEQ ID NO: 14).

In one embodiment, the EPCAM scFv of the dual CAR comprises the same VL as those of UBS-54 (SEQ ID NO: 15).

In one embodiment, the light chain variable domain (VL) of EPCAM scFv of the dual CAR has the amino acid sequence of SEQ ID NO: 16.

In one embodiment, the EPCAM scFv of the dual CAR comprises the same VH and VL as those of UBS-54.

In one embodiment, the EPCAM scFv of the dual CAR comprises the same VH sequence as those of UBS-54, except CDR-H3 has one amino acid variation and has the amino acid sequence of DPFLHA, i.e., VH has the amino acid sequence of SEQ ID NO: 17.

In one embodiment, the EPCAM scFv of the dual CAR comprises the same VH sequence as those of UBS-54, except CDR-H3 has one amino acid variation and has the amino acid sequence of DPFLHL, i.e., VH has the amino acid sequence of SEQ ID NO: 18.

In one embodiment, the EPCAM scFv of the dual CAR comprises the same VH sequence as those of UBS-54, except CDR-H3 has one amino acid variation and has the amino acid sequence of DPFLHV, i.e., VH has the amino acid sequence of SEQ ID NO: 19.

In one embodiment, the EPCAM scFv of the dual CAR comprises the same VH sequence as those of UBS-54, except CDR-H3 has one amino acid variation and has the amino acid sequence of APFLHY, i.e., VH has the amino acid sequence of SEQ ID NO: 20.

In one embodiment, the EPCAM scFv of the dual CAR comprises the same VH sequence as those of UBS-54, except CDR-H3 has one amino acid variation and has the amino acid sequence of DPFAHY, i.e., VH has the amino acid sequence of SEQ ID NO: 21.

In one embodiment, the EPCAM scFv of the dual CAR comprises the same VH and VL sequences as those of UBS-54, except CDR-H3 has one amino acid variation and has the amino acid sequence of DPFLHF, i.e., VH has the amino acid sequence of SEQ ID NO: 22.

The preparation of EpCAM antibodies having different CDR3 of VH is shown in U.S. Provisional Application No. 63/009,018, or its PCT publication WO 2021/211510, which is incorporated herein by reference in its entirety.

U.S. Pat. No. 10,428,136 discloses that different I domain mutants provide CARs with different affinity to ICAM-1; the '136 Patent is incorporated herein by reference in its entirety. For example, I domain mutants having one mutation at F292A (Kd 20 μM), F292S (Kd 1.24 μM), L289G (Kd 196 nM), F265S (Kd 145 nM), and F292G (Kd 119 nM), or having two mutations at K287C/K294C (Kd 100 nM) in the wild-type I domain are suitable for the present invention. The above numbering of the amino acid residues is in reference to the amino acid sequence of the mature α_{L} integrin of SEQ ID NO: 1, which residue number 1 corresponds to the amino acid residue 26 of GenBank Accession No. NP_002200.

In one embodiment, the I domain in the bispecific CAR has the sequence of 130-310 amino acids of SEQ ID NO: 1, with one mutation of F292A, F292S, L289G, F265S, and F292G, or with two mutations at K287C/K294C.

The CAR of the present invention comprises a transmembrane domain which spans the membrane. The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor a or R chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. In preferred embodiments, the transmembrane domain is derived from CD28 or CD8, which give good receptor stability.

The CAR of the present invention comprises one or more co-stimulatory domains selected from the group consisting of human CD28, 4-1BB (CD137), ICOS-1, CD27, OX 40 (CD137), DAP10, and GITR (AITR). In one embodiment, the CAR comprises two co-stimulating domains of CD28 and 4-1BB.

The endodomain (the activating domain) is the signal-transmission portion of the CAR. After antigen recognition, receptors cluster and a signal is transmitted to the cell. The most commonly used endodomain component is that of CD3-zeta (CD3 Z or CD3ζ), which contains 3 ITAMs. This transmits an activation signal to the T cell after antigen is bound. CD3-zeta may not provide a fully competent activation signal and additional co-stimulatory signaling may be needed. For example, one or more co-stimulating domains can be used with CD3-Zeta to transmit a proliferative/survival signal.

The CAR of the present invention may comprise a signal peptide N-terminal to the I domain so that when the CAR is expressed inside a cell, such as a T-cell, the nascent protein is directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. As an example, the signal peptide may derive from human CD8 or GM-CSF, or a variant thereof having 1 or 2 amino acid mutations provided that the signal peptide still functions to cause cell surface expression of the CAR.

The CAR of the present invention may comprise a spacer sequence as a hinge to connect scFv of EpCAM antibody or I domain with the transmembrane domain and spatially separate antigen binding domain from the endodomain. A flexible spacer allows to the binding domain to orient in different directions to enable its binding to a tumor antigen. The spacer sequence may, for example, comprise an IgG1 Fc region, an IgG1 hinge or a CD8 stalk, or a combination thereof. A human CD28 or CD8 stalk is preferred.

The present invention provides a nucleic acid encoding the CAR described above. The nucleic acid encoding the CAR can be prepared from an amino acid sequence of the specified CAR by a conventional method. A base sequence encoding an amino acid sequence can be obtained from the aforementioned NCBI RefSeq IDs or accession numbers of GenBenk for an amino acid sequence of each domain, and the nucleic acid of the present invention can be prepared using a standard molecular biological and/or chemical procedure. For example, based on the base sequence, a nucleic acid can be synthesized, and the nucleic acid of the present invention can be prepared by combining DNA fragments which are obtained from a cDNA library using a polymerase chain reaction (PCR).

The nucleic acid encoding the CAR of the present invention can be inserted into a vector, and the vector can be introduced into a cell. For example, a virus vector such as a retrovirus vector (including an retrovirus vector, a lentivirus vector, and a pseudo type vector), an adenovirus vector, an adeno-associated virus (AAV) vector, a simian virus vector, a vaccinia virus vector or a Sendai virus vector, an Epstein-Barr virus (EBV) vector, and a HSV vector can be used. As the virus vector, a virus vector lacking the replicating ability so as not to self-replicate in an infected cell is preferably used.

For example, when a retrovirus vector is used, the process of the present invention can be carried out by selecting a suitable packaging cell based on a LTR sequence and a packaging signal sequence possessed by the vector and preparing a retrovirus particle using the packaging cell. Examples of the packaging cell include PG13 (ATCC CRL-10686), PA317 (ATCC CRL-9078), GP+E-86 and GP+envAm-12, and Psi-Crip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies.

The present invention provides T cells or natural killer cells (NK cells) modified to express the bispecific CAR as described above. CAR-T cells or CAR-NK cells of the present invention bind to EpCAM-1 and ICAM-1 via the anti-EpCAM or I domain of CAR, thereby a signal is transmitted into the cell, and as a result, the cell is activated. The activation of the cell expressing the CAR is varied depending on the kind of a host cell and an intracellular domain of the CAR, and can be confirmed based on, for example, release of a cytokine, improvement of a cell proliferation rate, change in a cell surface molecule, killing target cells, or the like as an index.

T cells or NK cells modified to express the bispecific CARs can be used as a therapeutic agent for a disease. The therapeutic agent comprises the T cells expressing the bispecific CAR as an active ingredient and may further comprise a suitable excipient. Examples of the excipient include pharmaceutically acceptable excipients known to a person skilled in the art.

The present invention further provides an adoptive cell therapy method for treating cancer. The method comprises the steps of: administering the bispecific CAR-T cells or bispecific CAR-NK cells of the present invention to a subject suffering from cancer, wherein the cancer cells of the subject overexpress EpCAM or express the inducible ICAM-1, and the CAR-T cells or CAR-NK cells bind to cancer cells to kill the cancer cells. Cancers suitable to be treated by the present invention include, but not limited to thyroid cancer, gastric cancer, pancreatic cancer, and breast cancer.

The simultaneous targeting of two tumor antigens has one major shortcoming that it may significantly elevate the on-target off-tumor side effects. The bispecific CARs of the present invention use two lower-affinity CARs, and they are restricted to recognize tumors cells expressing high-density antigens, whereas non-malignant tissues with low levels of antigen expression are spared.

The low affinity dual CARs of the present invention are particularly useful against heterogenous tumors. Upon encountering either EpCAM⁺ICAM-1⁻, EpCAM⁻ICAM-1⁺, or EpCAM⁺ICAM-1⁺ target cells, dual CAR T cells secret pro-inflammatory cytokines in the microenvironment, which further upregulate ICAM-1 in tumor cells through IFN-γ and TNF-α signaling pathways. EpCAM⁻ICAM-1⁺ cells that are able to escape EpCAM single CAR can now be recognized and eradicated by dual CAR through the ICAM-1 targeting, thereby preventing EpCAM-negative or EpCAM-low relapses. Even in EpCAM⁺ICAM-1⁺ tumors, simultaneously targeting EpCAM and ICAM-1 with low-affinity dual CAR renders CAR T cells less susceptible to immune suppression, resulting in more durable tumor remission. Given that ICAM-1 can be induced upon CAR T cell localization and activation, and thus become targetable, the dual CARs augment eradication of tumors that are ICAM-1 low or negative at the time of diagnosis.

The low affinity dual CARs of the present invention target both EpCAM and ICAM-1 and reduce the likelihood of tumor relapse and maintain long-term tumor-free remission. The present dual CAR therapy can be combined other approaches such as PD1/PD-L1 checkpoint inhibitors^{50, 51}, disruption of PD-1-PD-L1 and CTLA4 pathways⁵²⁻⁵⁴, deletion of TGF-β receptor II (TGFβR2) to suppress T_{reg} conversion⁵⁵, as well as armoring CAR T cells to deliver stimulatory cytokines (e.g., IL-12, IL-15 and IL-18)⁵⁶⁻⁵⁸, to enhance T cell functionality and reduce immune escape.

The bispecific low affinity CARs of the present invention overcome antigen escape and alleviate on-target off-tumor toxicities. The combined activity of EpCAM- and ICAM-1-specific CARs results in a synergistic clearance of heterogeneous tumors and a reduced occurrence of tumor relapse.

This application demonstrates that lower-affinity UBS54 CAR approaching micromolar KD is able to robustly and durably eradicate multiple difficult-to-treat solid tumors without triggering severe treatment-related toxicities. However, UBS54 CAR T alone is susceptible to relapse of EpCAM positive solid tumors and fails to completely eliminate tumors with heterogeneous EpCAM expression in gastric cancer models. In contrast, bispecific dual CAR T cells that express both lower-affinity EpCAM CAR and affinity-tuned I domain CAR enhance anti-tumor activity and reduce the rate of tumor relapse. Additional targeting of ICAM-1 significantly elevates tumor response to CAR T cells even when tumors have little ICAM-1 expression prior to treatment. ICAM-1 can be induced by proinflammatory cytokines secreted upon CAR interaction with the primary antigen, EpCAM, rendering tumor cells more susceptible to bispecific dual CAR T cells.

Our data demonstrate that EpCAM-specific CAR T cells alone were able to robustly eradicate multiple solid tumors but were susceptible to relapse. In contrast, bispecific dual CAR T cells that additionally targeted ICAM-1 prevent tumors from becoming resistant or reducing relapse of tumors with heterogeneous EpCAM expression. The addition of ICAM-1 targeting significantly elevated the antitumor activity of CAR T cells, even when tumors had little ICAM-1 expression prior to treatment. Our data show that ICAM-1 can be induced by proinflammatory cytokines secreted upon CAR interaction with the primary antigen, that is, EpCAM, rendering tumor cells more vulnerable to the dual CAR T cells.

The following examples further illustrate the present invention. These examples are intended merely to be illustrative of the present invention and are not to be construed as being limiting.

### EXAMPLES

### Materials and Methods

### Example 1. Cell Lines and Primary Human T Cells

The human glioblastoma cell line U-251 was provided by B. Law at Weill Cornell Medicine and was cultured in Dulbecco's Modified Eagle's Medium (DMEM, Corning) supplemented with 10% fetal bovine serum (FBS). Gastric cancer cell line SNU-638 was obtained from the Korean Cell Line Bank (Seoul National University, Seoul, Korea) and was cultured in RPMI-1640 (Corning) supplemented with 10% FBS. The human breast cancer cell lines MDA-MB-231 and SK-BR-3, pancreatic cancer cell lines SW1990 and Capan-2, colon cancer cell line HT-29, and gastric cancer cell line MKN-45 were purchased from the American Type Culture Collection (ATCC). MDA-MB-231 and SW-1990 were cultured in DMEM containing 10% FBS; SK-BR-3, Capan-2 and HT-29 were maintained in McCoy's 5A (ATCC) containing 10% FBS; and MKN-45 was maintained in RPMI-1640 supplemented with 10% FBS. All tumor cells were transduced with a Firefly Luciferase-F2A-GFP (FLuc-GFP) lentivirus (Biosettia) for bioluminescence-based cytotoxicity and mouse imaging experiments. All cells were cultured in a humidified incubator at 37° C. with 5% C02, and were routinely tested for mycoplasma using a MycoAlert™ detection kit (Lonza). Human leukopaks were commercially obtained from Biological Specialty Corporation, and were sorted for CD4/CD8-positive leukopak cells upon delivery. Primary human T cells were cultured in complete T cell growth medium: TexMACS medium (Miltenyi Biotec) containing 5% human AB serum (Sigma), 12.5 ng/mL IL-7 (Miltenyi Biotec), and 12.5 ng/mL IL-15 (Miltenyi Biotec).

### Example 2. Lentiviral Vector Construction

We designed two CARs containing scFvs derived from two anti-EpCAM monoclonal antibodies, C215 or UBS54, respectively. Genetic sequences of scFvs were inserted at the N-terminus of the 2^{nd} generation CAR architecture following this pattern from the 5′-LTR end: EF1α promoter, signal sequence, Myc tag, scFvs, CD8 hinge, transmembrane and cytoplasmic domains of CD28, and the cytoplasmic domain of CD3ζ molecule. The PET reporter gene SSTR2 was incorporated at the C-terminus following a “self-cleaving” ribosome-skipping porcine teschovirus-1 2 A (P2A) sequence.

For the bicistronic dual CAR, UBS54 scFv was cloned into the CD28-CD3(2nd generation CAR format, and LFA-1 I domain (F292A) was cloned into the 4-1BB-CD3(2nd generation CAR format. Both CARs were co-expressed in a tricistronic lentiviral vector with SSTR2 via T2A and P2A ribosome-skipping sequences.

For the tandem dual CAR, UBS54 scFv and LFA-1 I domain (F292A) were linked with a spacer (G4S)₂, and fused to a costimulatory domain (either CD28 or 41BB) and CD3ζ. The full CAR inserts were then ligated into a 3^{rd} generation pLenti backbone (VectorBuilder Inc., Chicago, Ill., USA)³³.

### Example 3. CAR T Cell Manufacturing

Lentivirus was packaged by VectorBuilder (Chicago, Ill., USA) and frozen at −80° C. until use. Jurkat T cells were transduced by an overnight incubation with lentivirus. Primary human T cells were transduced twice at 24 and 48 hours after activation with human T-activator CD3/CD28 Dynabeads (Gibco) at a bead-to-cell ratio of 1:1. T cells were maintained at 1-3×10⁶ cells/ml in complete T cell growth medium on a tube roller (Thermo Scientific) at 5 rpm. Transduction efficacy was evaluated by flow cytometry on day 6-7 after initial T cell activation. On day 10, cell products were cryopreserved in a 1:2 mixture of T cell complete growth medium and CS10 (STEMCELL) for in vitro and in vivo experiments.

### Example 4. Flow Cytometry

Flow cytometry data were acquired on a Gallios flow cytometer (Beckman Coulter Inc.) and analyzed using the FlowJo software (Tree Star Inc.). Prior to staining, cells were washed with PBS containing 1% BSA and blocked with 200 μg/ml mouse IgG (Sigma-Aldrich, cat. no. 15381). Cell staining was conducted at room temperature or at 4° C. for 15 min. Tumor cell surface markers were determined with the following antibodies from BioLegend: PE-Cy7 anti-human CD326 (EpCAM) antibody (clone 9C4), and APC anti-human CD54 (ICAM-1) antibody (clone HA58). For CAR detection and T cell phenotyping, the following antibodies were used: FITC anti-c-myc antibody (Miltenyi Biotec, clone SH1-26E7.1.3), APC anti-human SSTR2 antibody (R&D systems, clone 402038), and anti-human PE-Cy5 CD3/PE CD4/FITC CD8 cocktail (BioLegend, clone UCHT1; RPA-T4; RPA-T8), APC anti-human CD127 (BioLegend, clone A019D5), Brilliant Violet 421m anti-human CD25 (BioLegend, clone BC96), PE-Cy7 anti-human CTLA4 (BioLegend, clone BNI3), APC anti-human TIM3 (BioLegend, clone F38-2E2) and Brilliant Violet 421™ anti-human PD1 (BioLegend, clone EH12.2H7). Calcein Blue (Sigma-Aldrich, cat. no. M1255) staining along with forward- and side-scatter gating was used to exclude dead cells.

### Example 5. Determination of CAR Affinities

A saturation binding assay was performed to determine the binding affinities of CAR molecules expressed on the surface of Jurkat T cells. Recombinant human EpCAM monomer protein (R&D systems, cat. no. 9277-EP) was conjugated with Alexa Fluor 647 using a labeling kit (ThermoFisher, cat. no. A20186). 5×10⁴ C215, UBS54 or wild type Jurkat T cells were added in triplicate to a 96-well plate, and washed with PBS containing 1% BSA. Cells were then stained at 4° C. for 15 min with 2-fold serially diluted Alexa Fluor 647-conjugated EpCAM protein starting from 1 μM. Flow cytometry was performed and the mean fluorescence intensities (MFI) were used to calculate K_{D} values using the one-site nonlinear regression model (GraphPad Prism 8).

### Example 6. Bioluminescence-Based Cytotoxicity Assay

5×10³ firefly luciferase-expressing tumor target cells were co-cultured with either non-transduced (NT) or CAR T cells in 96-well plates at indicated effector to target (E:T) ratios. Co-cultures were performed in T cell growth media containing 150 μg/ml D-luciferin (Gold Biotechnology) without any cytokine supplement. Luminescence was measured by a microplate reader (TECAN Infinite M1000PRO) at varying time points. The percentage of viability was calculated by dividing relative light units (RLU) by that of target cells only. The percent lysis was calculated by the following equation: percentage=[(target cells only RLU−test RLU)/target cells only RLU]×100. Data were presented as the mean±s.d. for the triplicate wells.

### Example 7. CRISPR-Cas9 Editing of Cell Lines

EpCAM knockout cell lines were generated using the Alt-R CRISPR-Cas9 System (Integrated DNA Technologies, Inc, IA, USA) according to the manufacturer's instructions. TracrRNA and crRNA oligos were annealed in equimolar concentrations by heating at 95° C. for 5 min, followed by gradual cooling to room temperature. Two crRNAs were used to target two exons of EpCAM: crRNA-AA, 5′-rGrArU rCrArC rArArC rGrCrG rUrUrA rUrCrA rArCrG rUrUrU rUrArG rArGrC rUrArU rGrCrU-3′; crRNA-AB, 5′-rGrUrG rCrArC rCrArA rCrUrG rArArG rUrArC rArCrG rUrUrU rUrArG rArGrC rUrArU rGrCrU-3′. Guide RNA duplex (22 pmol) was then incubated with Cas9 nuclease (18 pmol) at room temperature for 20 min to form ribonucleoprotein (RNP) complex. 5×10⁴ cells were mixed with RNP-AA and RNP-AB complexes into a 10 μl Neon tip. After electroporation (1250V/20 ms/3 pulses) using the Neon™ Transfection System (Invitrogen, CA, USA), cells were immediately transferred to a 24-well plate containing 0.5 ml of pre-warmed culture medium, and incubated in a humidified 37° C., 5% CO₂ incubator. 5 days later, EpCAM expression on cell surface was assessed by flow cytometry.

### Example 8. Cytokine Analysis

5×10³ non-transduced (NT) or CAR T cells were co-cultured with EpCAM-positive or EpCAM-negative target cells in 96-well plates at an E:T ratio of 1:1. After 24 h of incubation at 37° C., culture supernatants were collected for cytokine detection by Bio-Plex MAGPIX (Bio-Rad). Mouse plasma was harvested and stored at −80° C. for cytokine analysis.

### Example 9. In Vivo Mouse Studies

4- to 6-week-old male NOD-scidIL2Rg^{null} (NSG) mice were purchased from the Jackson Laboratory, and housed in the Animal Core Facility at Weill Cornell Medicine. All experiments were performed in accordance with the Institutional Animal Care and Use Committee (IACUC) guidelines at Weill Cornell Medicine. Peritoneal gastric cancer models were established by injecting 0.5×10⁶ firefly luciferase (FLuc)-expressing SNU-638 tumor cells into the peritoneal cavity. After 7 days, non-transduced control T cells (NT), and anti-EpCAM C215 and UBS54 CAR T cells (10×10⁶/mouse) were injected intraperitoneally. For systemic gastric cancer models, both MKN-45-FLuc⁺ tumor cells (0.5×10⁶/mouse) and T cells (10×10⁶/mouse) were injected via tail vein. T cells were administered 5 days after tumor inoculation. The orthotopic pancreatic tumor models were established by surgical implantation of Capan2-FLuc⁺ cells at a density of 0.1×10⁶ cells in 25 μL of 1:1 mixture of McCoy's 5A and Matrigel (Corning). Fifteen days later, T cells were injected intravenously via tail vein (10×10⁶/mouse). All T cells were cryopreserved and used for injection freshly after thawing. Tumor growth was monitored weekly using an IVIS® Spectrum in vivo imaging system (PerkinElmer). Bioluminescence images were acquired 15 minutes after intraperitoneal injection of 200 μL of 15 mg/mL D-luciferin (GoldBio). For peritoneal SNU-638 tumor model, D-luciferin was injected subcutaneously. Whole-body bioluminescence flux was used to estimate tumor burden. PET/CT imaging was performed to track T cell biodistribution using a micro-PET/CT scanner (Inveon, Siemens) 2 hours after intravenous injection of ¹⁸F-NOTA-OCT tracer (1,4,7-Triazaclononane-1,4,7-triacetic acid-octreotide).

For patient-derived xenograft model, human gastric tumors were mechanically dissected and subcutaneously transplanted into immunodeficient NSG mice. PO tumors were harvested, resected, and passaged to next generation of NSG mice. Seven days after inoculation of P3 tumors, mice were treated with 10×10⁶ NT or CAR T cells via tail vein. Tumor volume (V) was measure with a caliper on a weekly basis, and calculated using the formula V=[length×(width)²]/2.

To mimic heterogenous antigen expression, a mixture of SNU-638 (75% wild-type, 25% EpCAM knockout) or MKN-45 (90% EpCAM positive, 10% EpCAM knockout) tumor cells (1×10⁶/mouse) were implanted subcutaneously into the upper left flank of NSG mice. Five or seven days later, mice were randomly treated with 10×10⁶ non-transduced T cells, UBS54 CAR T cells, ICAM-1 CAR T cells, or bicistronic dual CAR T cells, or tandem dual CAR T cells via intravenous injection. Weekly bioluminescence imaging, tumor volume measurements, and PET/CT imaging was performed as described above. Mouse plasma was harvested and stored at −80° C. for cytokine analysis. Tumors were collected at the indicated time points to measure EpCAM and ICAM-1 expression by flow cytometry.

### Example 10. Cell Isolation from Tumors

Tumor tissues were cut into small pieces of 2-4 mm and digested at 37° C. for 1 h in 5 mL of RPMI 1640 medium supplemented with 10% FBS, 200 U/ml collagenase type IV (Gbico) and 100 U/ml DNase I (New England BioLabs Inc). Samples were then carefully triturated using a serological pipet and strained through a 70 m cell strainer to generate single-cell suspensions. Red blood cells were lysed in ACK lysis buffer (Lonza) for 5 min and excess debris were removed using debris removal solution (Miltenyi Biotec). Tumor-infiltrating lymphocytes were isolated by magnetic separation using human CD45 microbeads (Miltenyi Biotec). EpCAM and ICAM-1 expression on tumor cells as well as T cell phenotype were assessed by flow cytometry.

### Results

### Statistical Analysis

Unpaired, two-tailed student's t-test was performed to compare two groups, and in analyses where multiple-comparisons were required, ANOVA (one- or two-way) was used to evaluate statistical significance. When multiple groups were compared over multiple time points, statistical significance was determined by two-way ANOVA with Tukey's multiple comparisons test, as indicated in the figure legends. Mouse survival curves were generated using the method of Kaplan-Meier, and the significance was analyzed with the log-rank (Mantel-Cox) test. All statistical analyses were performed using Prism 8 (GraphPad Inc.). Statistical significance is defined as follows: ns, not significant; *P<0.05; **P<0.01; ***P<0.001; ****P<0.0001.

### Example 11. CAR Expression and Affinity Determination

We developed two anti-EpCAM CAR constructs based on the 2^{nd} generation CAR backbone (CD28-CD3ζ) with the incorporation of scFvs derived from two anti-EpCAM monoclonal antibodies, C215 or UBS54 (FIG. 1, top panel), respectively. C215 is a mouse monoclonal antibody obtained by immunization of mice, whereas scFv of UBS54 was selected from a phage display library^{31, 32}. The CD4/CD8 sorted primary T cells were transduced with lentiviruses after stimulation with anti-CD3/CD28 dynabeads for 24 hours.

Primary T cells were transduced with C215 or UBS54 EpCAM CAR lentiviruses, and stained with FITC anti-c-Myc, APC anti-SSTR2 or anti-human PE-Cy5 CD3/PE CD4/FITC CD8 cocktail. Binding was analyzed by flow cytometry. In the CAR T cell products, approximately 45% and 70% of T cells expressed C215 or UBS54 CAR, respectively. Both CAR T cells had a CD4:CD8 ratio of approximately 1:1.

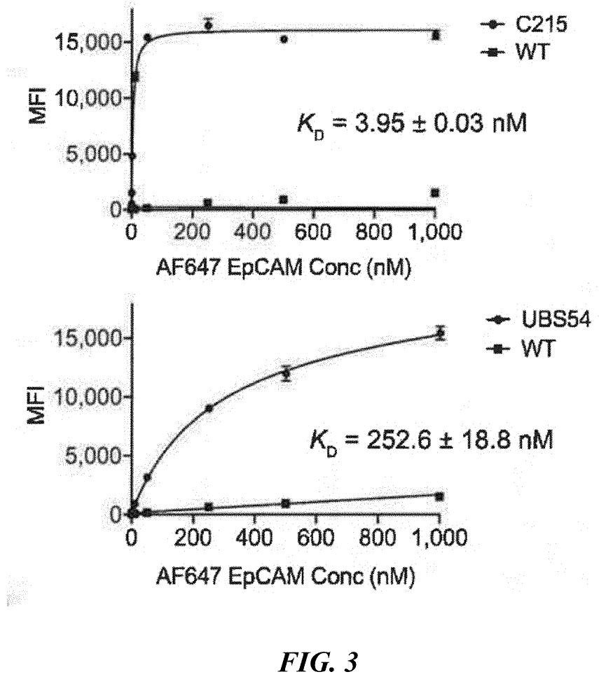

To compare the affinity of C215 and UBS54 CAR molecules, Jurkat T cells were transduced with a lentiviral vector and stained with serially diluted Alexa Fluor 647-conjugated EpCAM protein. The K_{D} value of UBS54 CAR (˜250 nM) was found to be substantially lower (>60-folds) than that of C215 CAR (˜4 nM) (FIG. 3).

### Example 12. Lower-Affinity EpCAM CAR Demonstrates Improved Cytolytic Index than Nanomolar Affinity CAR In Vitro

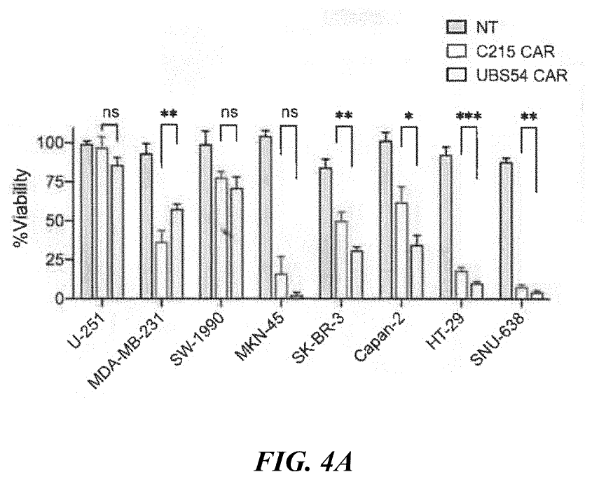

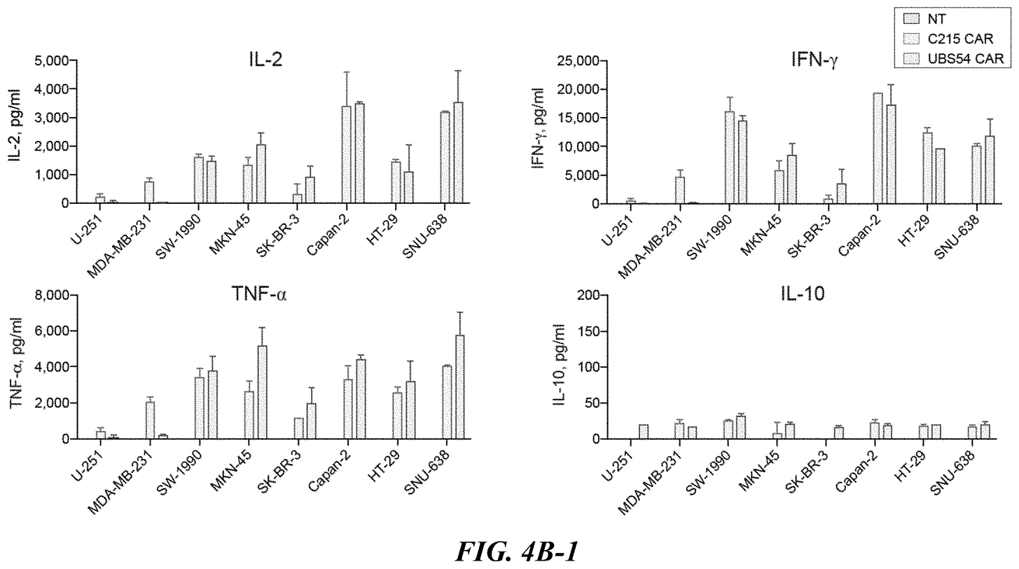

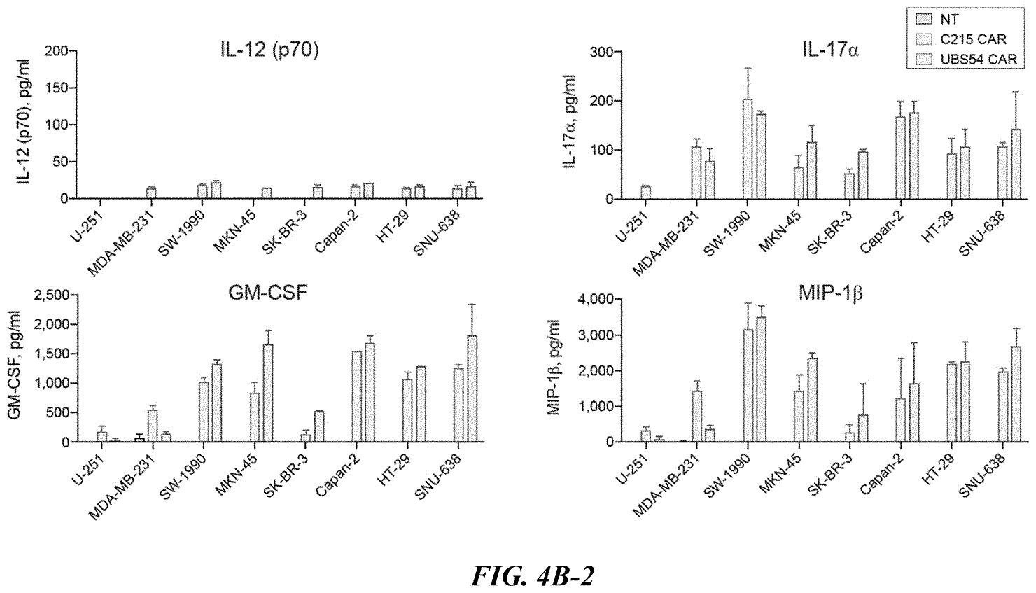

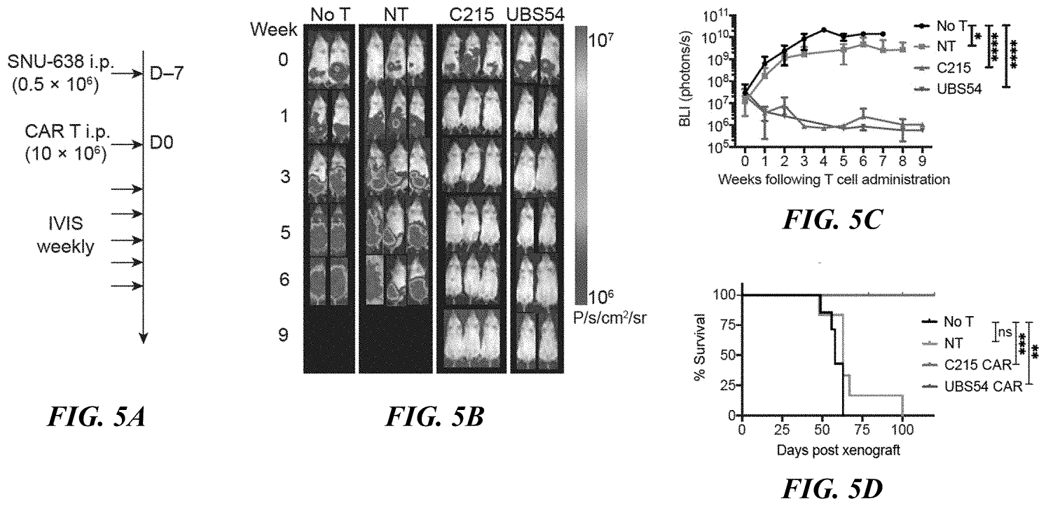

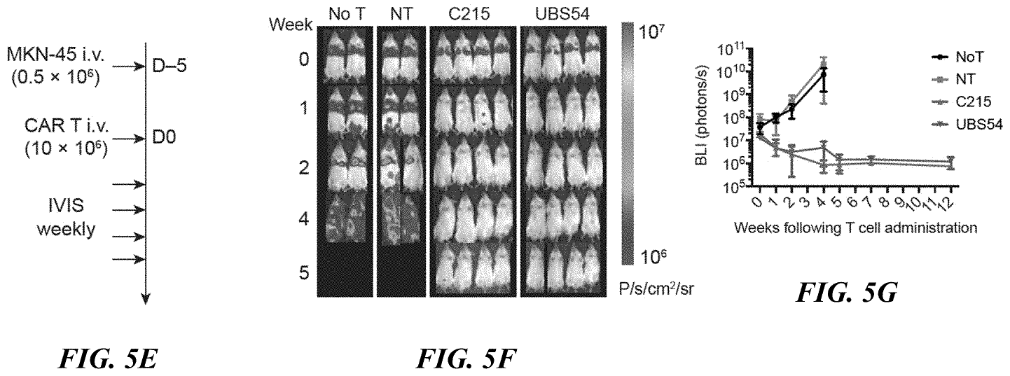

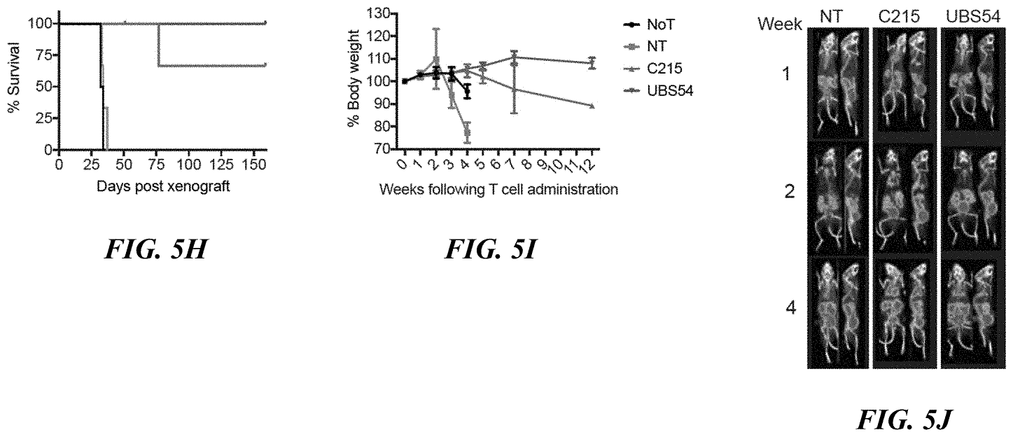

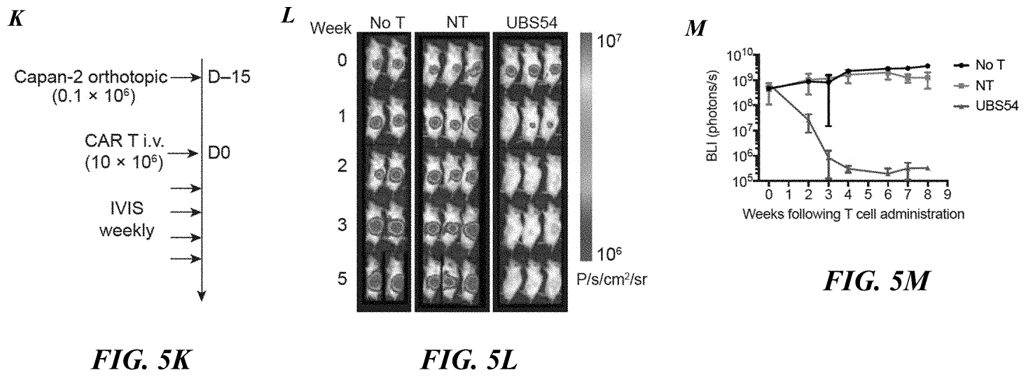

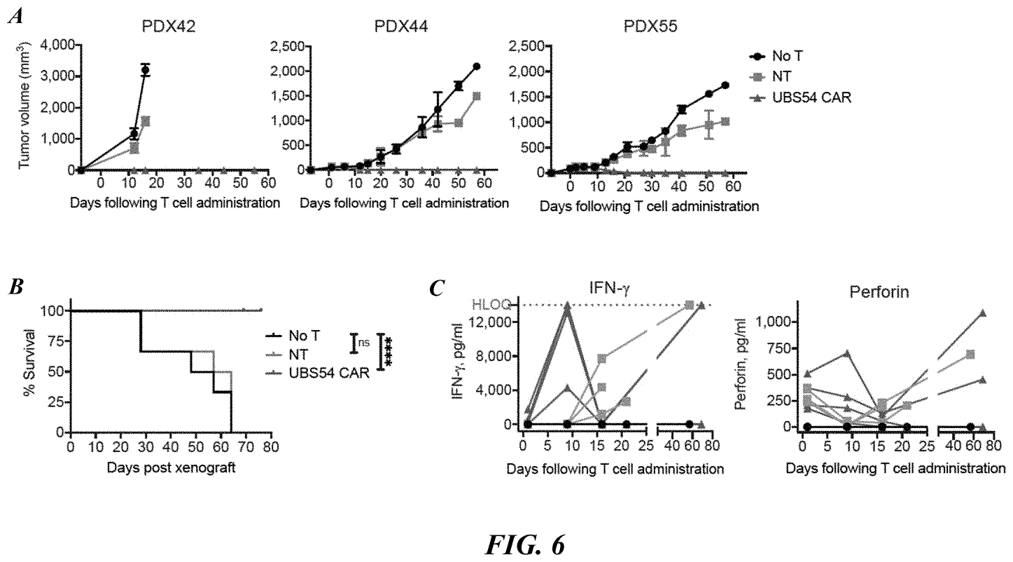

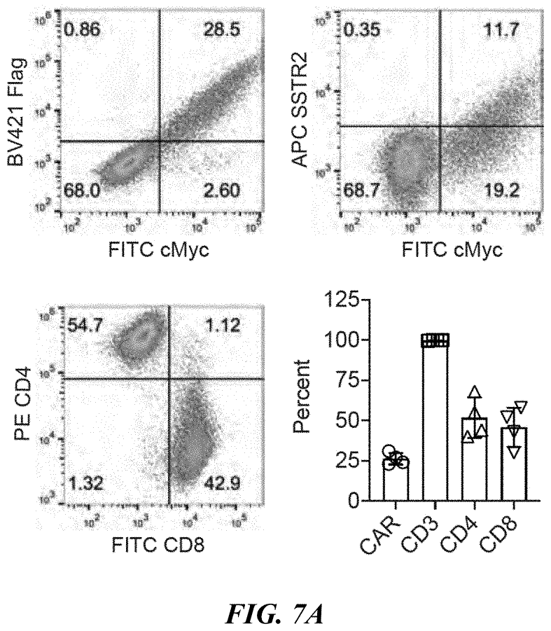

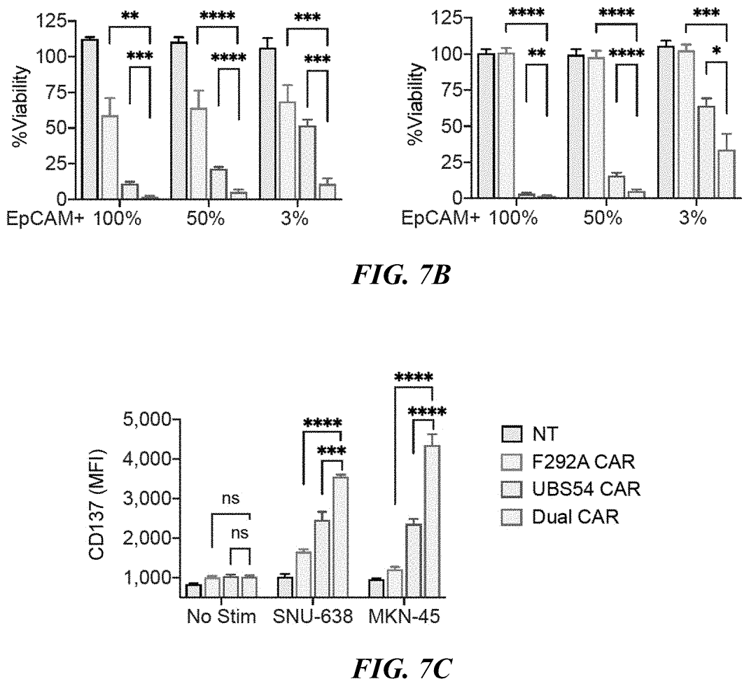

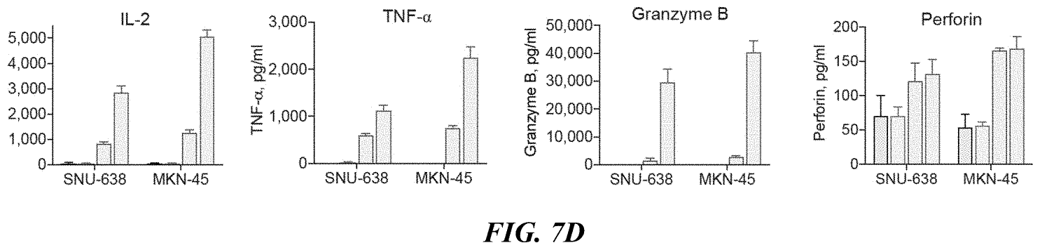

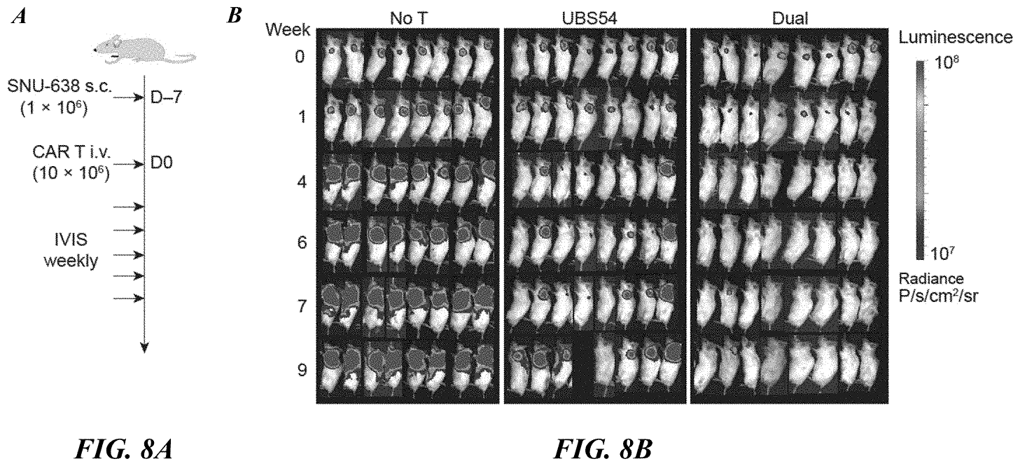

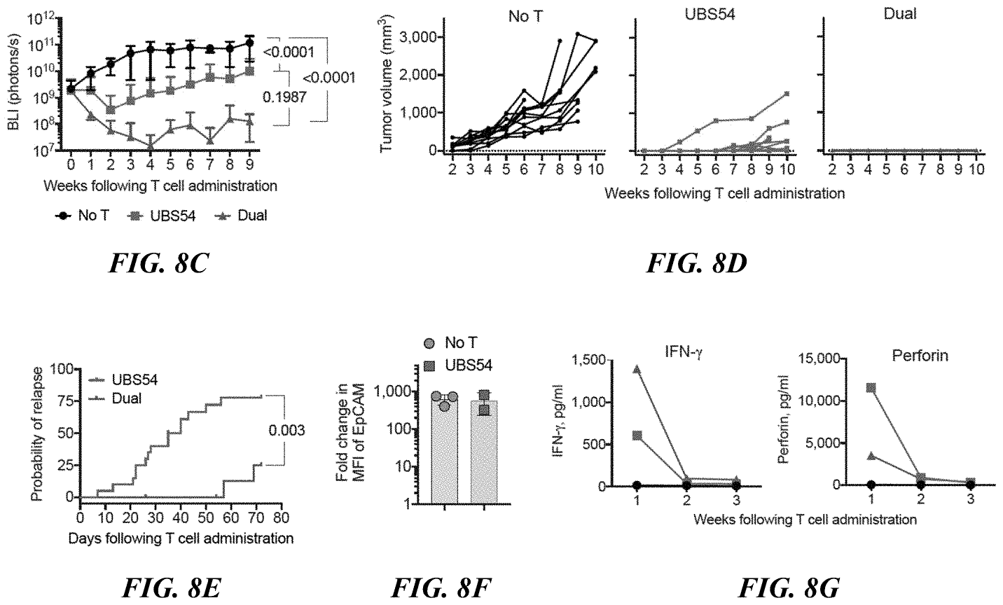

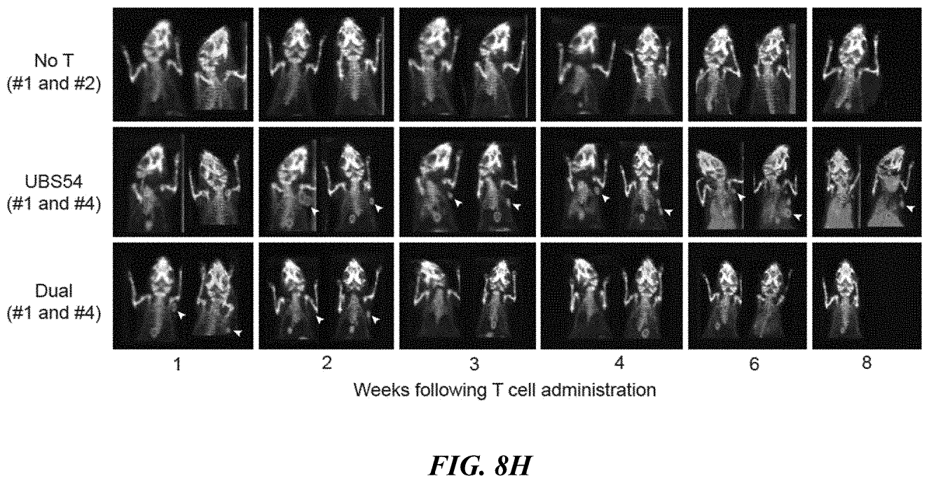

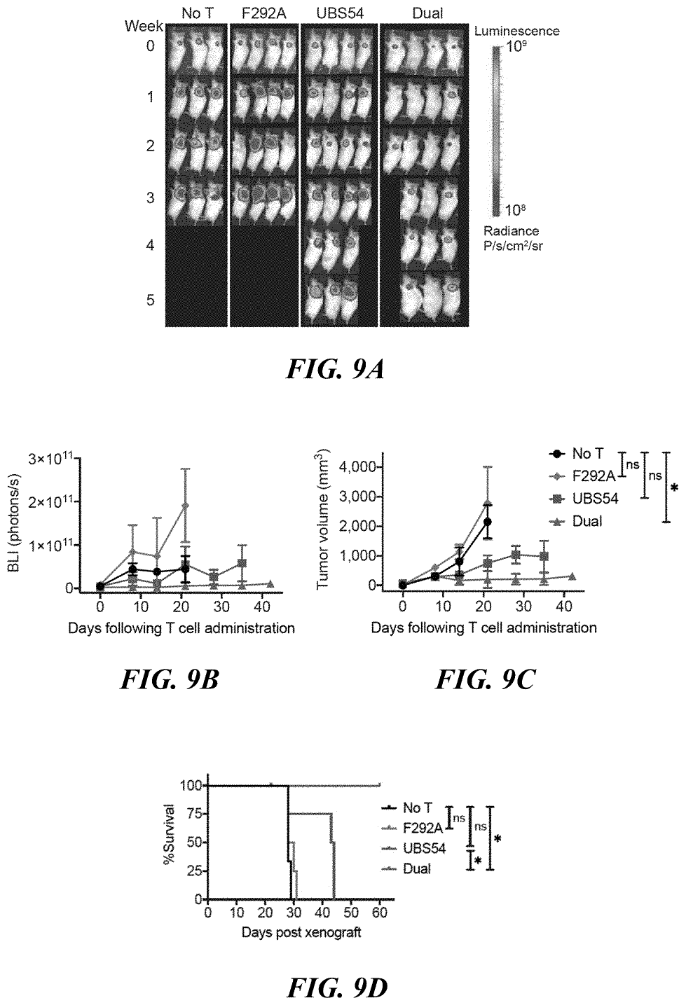

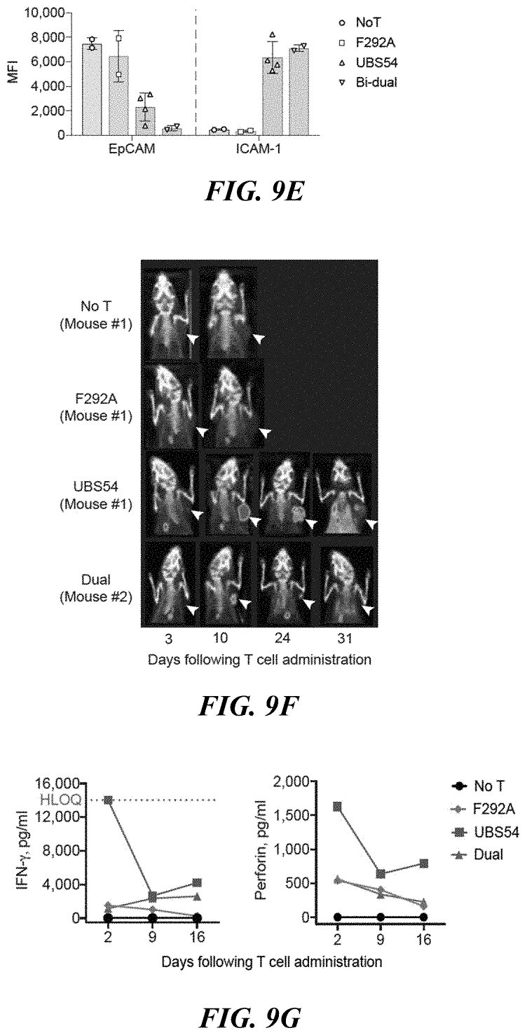

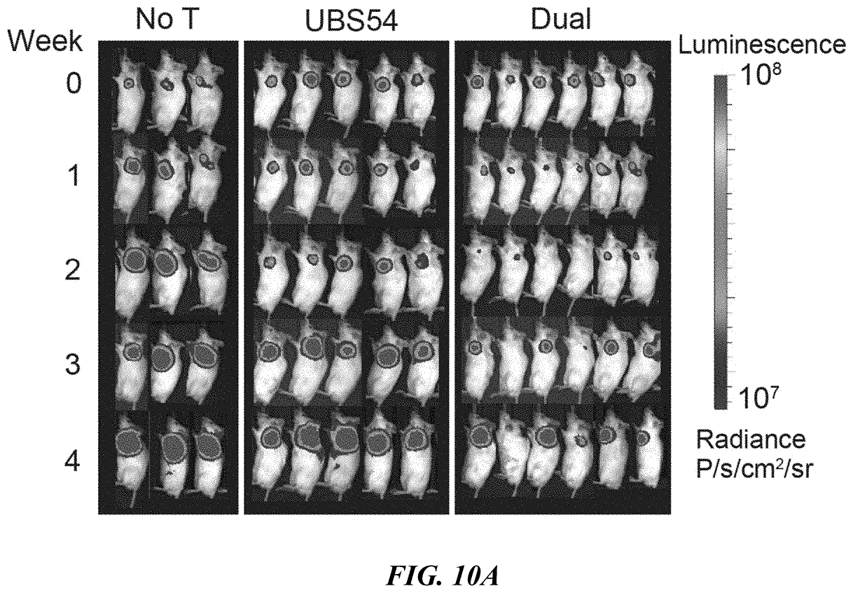

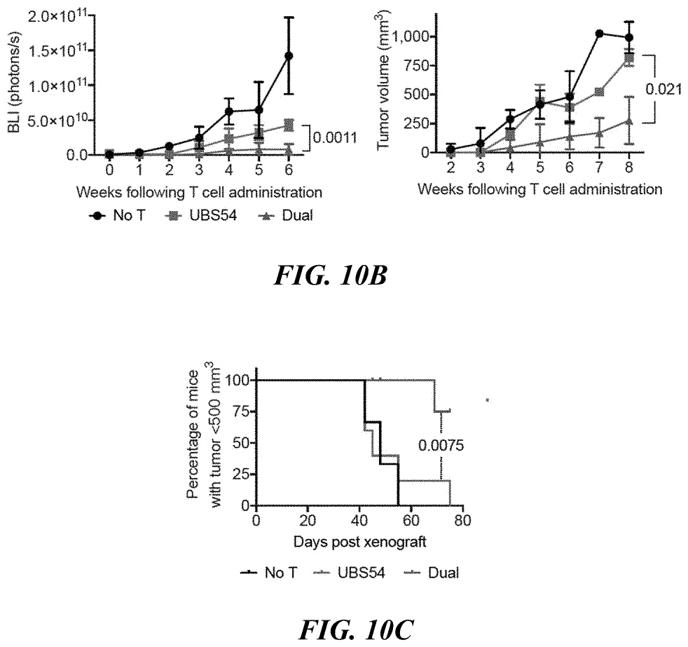

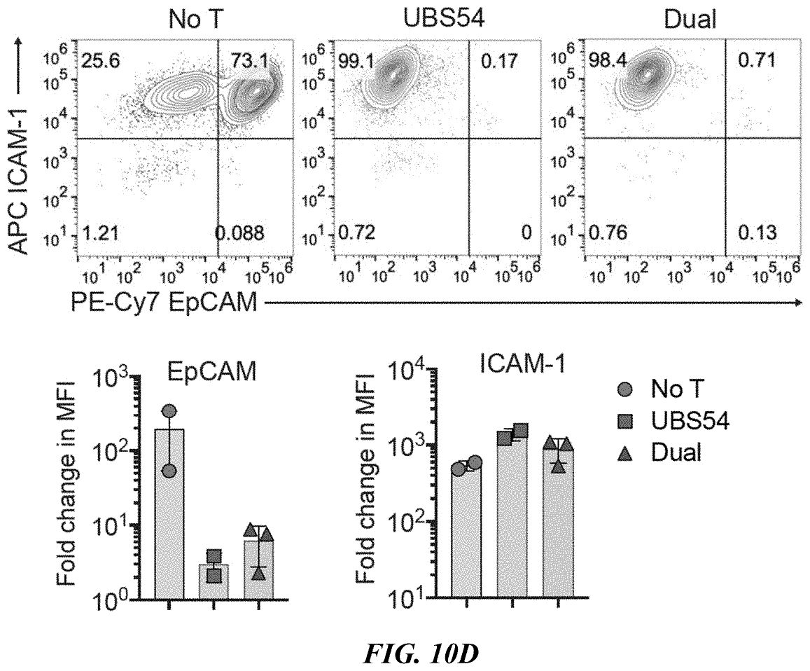

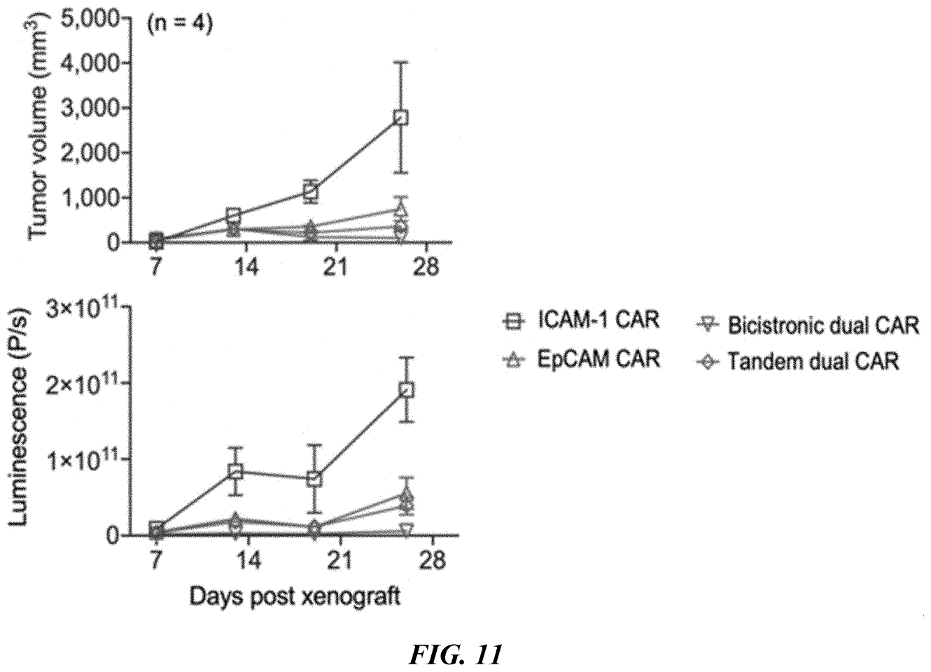

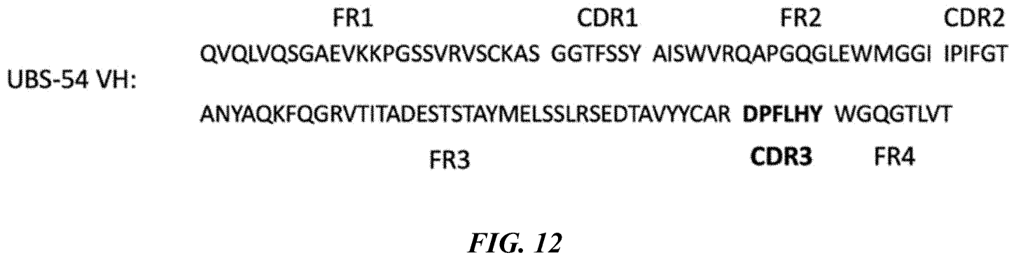

We tested the function of CAR T cells against a panel of tumor cell lines with a broad range of EpCAM expression as measured by flow cytometry. Surface expression of EpCAM in tumor cell lines as determined by flow cytometry after staining with PE-Cy5 anti-human EpCAM antibody. SK-BR3 (breast cancer), Capan-2 (pancreatic cancer), HT-29 (colon cancer), and SNU-638 (gastric cancer) overexpressed EpCAM, while MKN-45 (gastric cancer) showed moderate level of EpCAM expression, MDA-MB-231 (breast cancer) and SW-1990 (pancreatic cancer) expressed EpCAM at lower levels, and U-251 (glioblastoma) exhibited undetectable EpCAM surface expression. Cytolytic activity of CAR T cells was assessed by co-incubation of C125 or UBS54 CAR T cells with the panel of tumor target cells. UBS54 CAR T cells showed significantly greater cytotoxicity against target cells expressing high levels of EpCAM (SK-BR3, Capan-2, HT-29, and SNU-638), but mediated less killing of low-density EpCAM-expressing MDA-MB-231 compared to high-affinity C215 CAR T cells (FIG. 4A). Target cell lysis was generally EpCAM-dependent, evidenced by the lack of killing of EpCAM-negative U-251, as well as faster target killing as EpCAM surface density increased. One exception was MKN-45 which showed strong response to both C215 and UBS54 CAR T cells, despite having moderate level of EpCAM expression.

In response to stimulation with high-density EpCAM-expressing target cells, UBS54 CAR T cells secreted comparable to slightly higher levels of pro-inflammatory cytokines and chemokines (IL-2, IFN-γ, TNF-α, IL-17α, GM-CSF, and MIP-1β) compared to high-affinity C215 CAR T cells (FIG. 4B). Conversely, UBS54 CAR T cells produced less cytokines than C215 CAR T cells when co-cultured with low-density EpCAM-expressing MDA-MB-231 cells (FIG. 4B). Yet, in spite of high EpCAM expression in SK-BR-3 cells, both C215 and UBS54 CAR T showed lower levels of cytokine production when exposed to SK-BR-3. In all groups, less than 50 pg/ml of IL-12 (p70) and anti-inflammatory cytokine IL-10 were detected for both C215 and UBS54 CAR T cells. As expected, cytokine production by non-transduced T (NT) cells was undetectable against all target cells. Collectively, UBS54 CAR T cells possessing closer to micromolar affinity retained potent response to high-density EpCAM-expressing tumor targets, while displaying reduced reactivity to low-density EpCAM.

### Example 13. Lower-Affinity EpCAM CAR T Cells Eliminate Solid Tumors in Mouse Xenograft Models

After validation of CAR T specificity against cell lines in vitro, we then examined C215 and UBS54 CAR T activities in a peritoneal gastric cancer model using firefly luciferase (FLuc)-expressing SNU-638 tumor cells. SNU-638 is an intestinal type of gastric cancer cell line that shows microsatellite instability³⁴. This cell line has been used for screening anti-cancer drugs and in our previous study with ICAM-1 targeting CAR T cells^{35, 36}, NSG mice were xenografted intraperitoneally (i.p.) with SNU-638 cells and then treated 7 days later with NT or CAR T cells i.p. (FIG. 5A). Untreated mice (No T) showed continued tumor growth in the intestinal tract and peritoneal cavity, and expired within 60 days after tumor xenograft (FIGS. 5B-5D). NT cells slowed but did not control tumor growth, resulting in only marginal survival benefit. However, C215 and UBS54 anti-EpCAM CARs rapidly eliminated tumors within 1 week after a single dose of CAR T cells, and prevented tumor relapse through the end of the study (125 days post tumor xenograft) (FIGS. 5B-5C). All CAR T treated mice remained healthy and survived without any signs of treatment-related toxicities (FIG. 5D).

Next, we evaluated C215 and UBS54 CAR T cells in a systemic gastric cancer model with MKN-45 tumor cell line (FIG. 5E). MKN-45 was derived from a poorly differentiated gastric adenocarcinoma of medullary type, having the natures of both ordinary gastric mucosa and intestinal metaplastic mucosa³⁷. An intravenous injection of 0.5×10⁶ MKN-45 cells initially formed tumor lesions in the lung, and then the tumors quickly metastasized to liver, head, and joints of the animals. Untreated mice succumbed to the aggressive tumor growth approximately 30 days following tumor inoculation. Mice treated with NT cells had neither treatment effect nor survival benefit over No T cohort (FIGS. 5F-5H). In stark contrast, an equivalent number of CAR T cells led to complete tumor regression within one week after CAR T treatment, and no tumor recurrence was seen through 150 days post tumor implantation (FIGS. 5F-5H). We engineered CAR T cells to co-express human somatostatin receptor 2 (SSTR2) (FIGS. 5F-5H), which serves as a PET reporter and enables us to track CAR T cell distribution spatially and temporally using ¹⁸F-NOTA-Octreotide³⁸. PET/CT imaging of CAR T-treated mice showed above the background levels of tracer uptake in the lungs at 1-week after T cell infusion (1.5% ID/cm³ for C215 and 1.2% ID/cm³ UBS54 versus 0.8% ID/cm³ for NT) (FIG. 5J). The CAR T density in the lungs was relatively lower compared to the CAR T levels (approximately 3% ID/cm³) observed in our previous ICAM-1 CAR T studies³⁰, likely due to the ability of EpCAM CAR T cells to rapidly eliminate tumors in the lungs without excessive expansion. However, C215 CAR T appeared to continually expand in the lungs (5.2% ID/cm³ at 4 weeks), and other lymphoid organs, while UBS54 CAR T contracted at 2-weeks and began to expand at week 4 (2.4% ID/cm³). Unabating expansion of C215 CAR T after tumor elimination should be driven by GvHD, i.e., human TCR recognition of mouse tissue major histocompatibility complex (MHC) molecules, corroborated by typical signs of GvHD including a gradual loss of body weight and fur loss, and reduced mobility. Tumor-unrelated CAR T cell expansion was also seen in UBS54 CAR treated mice, but with a later onset (beginning from 4 weeks following T cell infusion) and to a lesser degree. The severity of GvHD and mortality was overall lower with UBS54 CAR T cohort.

Finally, the efficacy of lower-affinity EpCAM targeting UBS54 CAR T cells was evaluated against pancreatic tumors, which frequently overexpress EpCAM, by surgically implanting 0.1×10⁶ Capan2-FLuc⁺ cells into mouse pancreas (FIG. 5K). Similar to the response seen against gastric cancer models, infusion of UBS54 CAR T cells led to rapid and durable tumor elimination, while tumors continued to grow in NT and No T cohorts (FIGS. 5L-5M). Collectively, we demonstrated that UBS54 CAR T cells, approaching micromolar affinity and despite the 60-fold lower affinity compared to C215 CAR T, were highly effective in eliminating EpCAM-positive cancer cell lines implanted either intraperitoneally, systemically, or orthotopically into the pancreas.

### Example 14. UBS54 EpCAM CAR T Cells Eradicate Gastric Tumors in Patient-Derived Xenograft Models In Vivo

After observing durable and complete response of tumors to lower-affinity UBS54 CAR T cells in mouse xenograft models with cancer cell lines, we next assessed CAR T activity in patient-derived xenograft (PDX) models that more closely resemble clinical tumors. NSG mice were subcutaneously engrafted with gastric tumor specimens derived from three patients (PDX42, PDX44 and PDX55) that had moderate to strong levels of membranous and cytoplasmic EpCAM expression. One week later, mice in each PDX model were randomized and assigned to three treatment cohorts: no treatment, 10×10⁶NT, or 10×10⁶UBS54 CAR T. PDX42 tumors grew aggressively and reached a volume of 3,000 mm³ in approximately 23 days after tumor inoculation (FIG. 6A). Infusion of NT cells slowed but did not stop tumor growth, and mice succumbed to tumor 28 days after tumor inoculation. By contrast, a single dose of UBS54 CAR T suppressed the progression of aggressive PDX42 tumors, and produced 100% tumor-free survival (FIGS. 6A-6B). PDX44 and PDX55 tumors grew relatively slower, reaching a volume of 1,000 mm³ in approximately 45 days following tumor implantation. In stark contrast to the progressive tumor growth in No T and NT cohorts, PDX44 and PDX55 tumors showed fast response to UBS54 CAR and were completely eradicated by 15 days post T cell injection. The fast response to CAR T treatment was further corroborated by cytokine release following T cell infusion. High amounts of IFN-γ and perforin were detected in mouse serum from UBS54 CAR T-treated mice, peaking at one week after T cell infusion, and returning to background levels at the following week when tumors were completely eradicated (FIG. 6C). At approximately 10 weeks after treatment, 2 out of 4 tumor-free UBS54 CAR T-treated mice had a spike in serum cytokines, likely due to GvHD-driven T cell expansion. In NT-treated mice, serum cytokines stayed at basal or low levels within the first week following T cell injection, but showed a surge in cytokine at approximately 2 weeks (FIG. 6C). PET/CT imaging with a SSTR2 tracer revealed rapid accumulation of CAR T cells at the subcutaneous tumors, followed by subsequent contraction when tumors were eliminated on day 14. In the NT-treated mice, subcutaneous tumors were seen in the flank by CT images, and continued to grow with little tracer uptake. Taken together, these results further confirmed the potent anti-tumor activity of UBS54 CAR T against gastric tumors.

### Example 15. Dual Targeting of EpCAM and Inducible ICAM-1 with a Dual CAR Promotes Killing of Heterogenous Tumors In Vitro

Heterogeneous antigen expression, especially in solid tumors, is increasingly recognized as a cause of antigen-escape relapses and treatment failure. We generated CRISPR-Cas9 knockout tumor cell lines and mixed them with wild-type cell lines to mimic heterogeneity in EpCAM expression.

A mixture of EpCAM+ and EpCAM− SNU-638 or MKN-45 cells were treated with 10 ng/ml IFN-γ, or co-incubated with NT or UBS54 CAR T cells at an E:T ratio of 1:1. Surface EpCAM and ICAM-1 expression were evaluated by flow cytometry 24 hours later. After incubating a heterogeneous mixture of SNU-638 or MKN-45 cells (40-60% EpCAM⁺) with UBS54 CAR T cells for 24 hours, EpCAM⁺ cells were eliminated, whereas EpCAM⁻ tumor cells were largely spared. EpCAM expression in SNU-638 or MKN-45 remained unaltered either after incubation with NT cells or addition of IFN-γ. In comparison, ICAM-1 expression of both cell lines was significantly upregulated after incubation with CAR T cells. Notably, ICAM-1 expression in MKN-45 was significantly elevated either by IFN-γ or CAR T treatment, resulting in two distinct populations of intermediate and high ICAM-1 expression.

To broaden CAR T efficacy against tumor cells with heterogenous EpCAM expression, we designed a bicistronic dual CAR that incorporates sequences of UBS54 scFv and affinity-tuned LFA-1 I domain (F292A, 20 μM³⁰) into one lentiviral vector (FIG. 1, 3rd panel). Costimulatory domains of CD28 and 41BB were used for EpCAM and ICAM-1 specific CARs, respectively. T cells transduced with this dual CAR construct showed approximately 25% of UBS54 and F292A CAR expression, indicated by antibody binding to c-Myc and Flag tags, which were fused at the N-terminal to UBS54 and I domain (F292A), respectively (FIG. 7A).

We first measured the in vitro cytolytic activity of F292A, UBS54, and dual CAR T cells against SNU-638 or MKN-45 with a mixture of EpCAM⁺ and EpCAM⁻ cells (100%, 50%, or 3% EpCAM⁺). Micromolar affinity F292A (K_{D}≈20 μM) CAR induced 40% specific lysis of SNU-638 cells at 48 hours through interaction with ICAM-1, whereas it mediated zero killing of MKN-45 cells with low ICAM-1 (FIG. 7B). UBS54 CAR T cells lysed approximately 90% of SNU-638 and MKN-45 wild-type cells that are 100% EpCAM⁺. The amount of cell death caused by UBS54 CAR T against heterogenous population of these cell lines was significantly higher than the percent of EpCAM⁺ cells, e.g., ˜80% lysis of 50% EpCAM⁺ cells and 40-50% lysis of 3% EpCAM⁺ cells. Additional killing of EpCAM⁻ cells was not from non-specific activity of UBS54 CAR T but most likely from bystander killing effect caused by CAR T interaction with EpCAM⁺ cells. As expected, dual CAR T attained increased killing of SNU-638 cells compared to single CAR T cells (F292A or UBS54) due to its ability to interact with both EpCAM and ICAM-1 antigens. Such enhanced killing, however, was also observed against MKN-45, which is ICAM-1 low and was completely unreactive to ICAM-1 specific CAR T. Synergy of EpCAM and ICAM-1 targeting by dual CAR T against MKN-45 is likely due to the induction of ICAM-1 in MKN-45 cells after exposure to proinflammatory cytokines above the activation threshold of ICAM-1 specific CAR. Stronger T cell activation of dual CAR was further evidenced by the induction of surface CD137 expression and quantitative measurement of cytokine production, which were highest in dual CAR T cells (FIGS. 7C-7D). Our bispecific design of CARs incorporates two CARs independently encoding CD28 and 41BB costimulatory domains, while single costimulatory domain is used for single CAR T (CD28 for UBS54 and 41BB for F292A CAR). The greater cytotoxic activity and cytokine secretion of dual CAR T are likely from complementary and additive costimulatory signals through both CD28 and 41BB when CAR is engaged with two antigens.

### Example 16. Dual CAR T Provides a Superior Activity Against Tumors with Homogeneous Antigen Expression to Single CAR T

We examined the activity of single versus dual CAR T cells against the hard-to-treat subcutaneous SNU-638 tumor model. Mice were subcutaneously implanted with 1×10⁶ SNU-638 tumor cells, and 7 days later treated with 10×10⁶ UBS54 or dual CAR T cells (FIG. 8A). UBS54 CAR T cells were able to eliminate tumors at early time points (14 complete remission (CR), bioluminescence intensity (BLI)≤2×10⁸ photons/second of 20 at 3 weeks after CAR T infusion, 70%), but tumor relapse occurred frequently (8 CR at 4-6 weeks, 40%; 5 CR at 8 weeks, 25%; FIGS. 8B-E). In contrast, dual CAR T cells led to complete clearance of the tumors in 100% of the cases (10 CR of 10 at 3 weeks). After 6 weeks after T-cell infusion, 2 mice had tumor relapses, but the relapsed tumors remained small and stable, in contrast to the relapsed and rapidly progressing tumors in UBS54 CAR T cell-treated mice (FIGS. 8B-E). To examine whether antigen downregulation occurred in the relapsed tumors, we analyzed EpCAM expression in tumor cells by flow cytometry. The relapsed tumors from UBS54 CAR T cell-treated mice had comparable EpCAM expression to tumors from untreated mice (FIG. 8F), confirming that altered antigen expression was not the cause for tumor resistance in this model. In contrast to comparable activity of single and dual CAR T cells against SNU-638 in vitro, UBS54 single CAR T cells were more susceptible to relapse of EpCAM+ solid tumors in vivo, whereas dual CAR T cells with additional targeting of ICAM-1 produced a significantly augmented antitumor response.

Serum cytokines were also measured weekly during the first 3 weeks following T-cell administration. Serum IFNg and perforin peaked 1 week after T-cell infusion and dropped to background levels in the following weeks when tumors were eliminated (FIG. 8G). The dynamics of CAR T-cell distribution and expansion were also assessed by PET/CT imaging using 18F-NOTA-OCT (FIG. 8H). UBS54 CAR T peaked 2 weeks after T-cell infusion and gradually contracted or persisted over the next several weeks. In comparison, dual CAR T cells peaked earlier, and fully contracted by 3 weeks after T-cell infusion. The slower expansion and contraction kinetics of UBS54 CAR T cells were likely due to lingering interaction between CAR T cells and tumor cells. The systemic expansion of UBS54 CAR T cells outside of tumor implants was seen beginning 6 weeks after T-cell infusion (elevation of tracer uptake in lungs, thymus, and lymph nodes). In some mice with tumor relapse after treatment with UBS54 CAR T cells, CAR T-cell infiltration and expansion in tumors were apparent, indicating that tumor relapse was not attributable to poor infiltration and/or persistence but to potential T-cell dysfunction.

### Example 17. Additional Targeting of Inducible ICAM-1 Complements EpCAM CAR T Activity Against Tumors with EpCAM Heterogeneity

We next examined if additional targeting of ICAM-1 can complement EpCAM CAR T activity against tumors with heterogenous expression of EpCAM. To this end, we generated two gastric cancer tumor models by subcutaneously implanting a heterogeneous population of MKN-45 or SNU-638 tumor cells into NSG mice. MKN-45 model comprised tumors with 90% EpCAM expression with little ICAM-1 expression, while SNU-638 contained 75% EpCAM⁺ and 100% ICAM-1⁺ cells.

NSG mice were subcutaneously implanted with a heterogeneous population of MKN-45 cells (90% EpCAM-positive, 10% EpCAM-negative, 1×10⁶ cells/mouse) and 5 days later received F292A, UBS54 or dual CAR T cells CAR T cells (10×10⁶ cells/mouse) via tail vein injection.

Against MKN-45 model, UBS-54 CAR T cells slowed tumor progression compared to untreated cohort but failed to achieve tumor remission (FIGS. 9A-9D). As expected, F292A CAR T cells displayed no treatment effect against ICAM-1 negative MKN-45 tumors. In contrast, dual CAR T cells demonstrated long-term regression of established solid tumors and significant survival benefits (FIGS. 9A-9D). Three of four mice (75%) attained CR one week following treatment with dual CAR T cells, among which 1 mouse that maintained CR through the end of the study, while 2 mice developed tumor relapse.

Flow cytometry analysis of relapsed MKN-45 tumors revealed diminished EpCAM surface expression in mice treated with UBS54 or dual CAR T cells, whereas MKN-45 tumors after F292A CAR T treatment showed no difference in EpCAM expression compared to the surface profile of untreated tumors. Consistent with the induction of ICAM-1 in CAR T-treated MKN-45 cells in vitro, MKN-45 tumors harvested from UBS54 or dual CAR T cohorts were found to have substantially elevated expression of ICAM-1. (FIG. 9E)

PET/CT imaging of CAR T cells revealed a pattern of CAR T cell expansion and contraction at the tumor site, which has been noted to be a hallmark of CAR T elimination of tumor³⁸. However, in the setting of heterogenous tumors, such biphasic kinetics were not associated with tumor elimination indicated by persistent tumor mass by CT. This would rather indicate elimination of EpCAM positive tumors but failed to control EpCAM negative tumor growth. In comparison, dual CAR T cells demonstrated an enhanced anti-tumor response against heterogenous tumors, which also resulted in a lower degree of CAR T expansion. In the F292A CAR T cohort, no specific tracer uptake at the MKN-45 tumors was observed. (FIG. 9F)

Consistent with a larger degree of expansion for single CAR T compared to dual CAR T, the amount of IFN-γ and perforin was higher from the plasma collected from UBS54 mice (FIG. 9G).

### Example 18. EpCAM−ICAM-1 Dual CAR T Mediates Longer Lasting Remission in a Heterogeneous SNU-638 Tumor Model

The superior antitumor efficacy of dual CAR T cells was also observed in the heterogeneous SNU-638 tumor model, which was seeded with 90% EpCAM⁺ and 10% EpCAM⁻ cells. The SNU-638 tumor model had high surface expression of ICAM-1, as opposed to little basal ICAM-1 expression in the MKN-45 tumor model.

NSG mice were subcutaneously implanted with a heterogeneous population of SNU-638 cells (90% wildtype, 10% EpCAM-negative, 1×10⁶ cells/mouse) and treated 7 days later with UBS54 or dual CAR T cells (10×10⁶ cells/mouse) via tail vein injection.

Similar to the activity against MKN-45 heterogenous tumor, UBS54 CAR T cells retained partial killing of SNU-638 tumors up to 2 weeks after infusion, which then gradually lost the activity against relapsing tumors (FIG. 10A). A significant treatment effect was seen only with the dual CAR T cells (FIGS. 10A-10C). Both flow cytometry and IHC analysis showed that relapsing or resistant SNU-638 tumors harvested 5 to 10 weeks after treatment had complete loss of EpCAM expression after single or dual CAR T-cell treatments, indicating these tumors consisted mainly of EpCAM-SNU-638 cells (FIG. 10D). At the same time, we found higher abundance of T cells in EpCAM− tumors of dual CAR T cell-treated mice compared with T cells in UBS54 single CAR T cell-treated tumors. Despite a significantly higher activity of dual CAR T cells over single UBS54 CAR T cells against heterogenous SNU-638 tumors, the low affinity F292A CAR alone was likely to be insufficient to eliminate the remaining SNU-638 tumors that mainly contained EpCAM−ICAM-1⁺ cells.

### Example 19. Comparison of Different CAR Treatments

MKN-45 (90% EpCAM positive, ICAM-1 low) tumor cells (1×10⁶/mouse) were implanted subcutaneously into the upper left flank of NSG mice. Five or seven days later, mice were randomly treated with 10×10⁶ UBS54 CAR T cells, ICAM-1 (F292A) CAR T cells, or bicistronic dual CAR T cells, or tandon dual CAR T cells via intravenous injection (see Example 9). The response (tumor size and luminescence) to different treatments ((ICAM-1 CAR, EpCAM CAR, bicistronic dual CAR, and tandem dual CAR) are shown in FIG. 11. Due to low ICAM-1 expression, ICAM-1 CAR did not produce tumor killing. The EpCAM CAR, bicistronic dual CAR, and tandem dual CAR showed similar tumor killing.

### REFERENCES

1. Jackson, H. J., Rafiq, S. & Brentjens, R. J. Driving CAR T-cells forward. Nature Reviews Clinical Oncology 13, 370-383 (2016).
2. Sadelain, M., Rivière, I. & Riddell, S. Therapeutic T cell engineering. Nature 545, 423-431 (2017).
3. June, C. H. & Sadelain, M. Chimeric Antigen Receptor Therapy. New England Journal of Medicine 379, 64-73 (2018).
4. Maude, S. L. et al. Chimeric Antigen Receptor T Cells for Sustained Remissions in Leukemia. New England Journal of Medicine 371, 1507-1517 (2014).
5. Brudno, J. N. & Kochenderfer, J. N. Chimeric antigen receptor T-cell therapies for lymphoma. Nat Rev Clin Oncol 15, 31-46 (2018).
6. Brudno, J. N. et al. T Cells Genetically Modified to Express an Anti-B-Cell Maturation Antigen Chimeric Antigen Receptor Cause Remissions of Poor-Prognosis Relapsed Multiple Myeloma. Journal of Clinical Oncology 36, 2267-2280 (2018).
7. Cohen, A. D. et al. B cell maturation antigen-specific CAR T cells are clinically active in multiple myeloma. J Clin Invest 129, 2210-2221 (2019).
8. Park, J. H. et al. Long-Term Follow-up of CD19 CAR Therapy in Acute Lymphoblastic Leukemia. New EnglandJournal of Medicine 378, 449-459 (2018).
9. Sotillo, E. et al. Convergence of Acquired Mutations and Alternative Splicing of CD19 Enables Resistance to CART-19 Immunotherapy. Cancer Discovery 5, 1282 (2015).
10. Gardner, R. et al. Acquisition of a CD19-negative myeloid phenotype allows immune escape of MLL-rearranged B-ALL from CD19 CAR-T-cell therapy. Blood 127, 2406-2410 (2016).
11. Fry, T. J. et al. CD22-targeted CAR T cells induce remission in B-ALL that is naive or resistant to CD19-targeted CAR immunotherapy. Nat Med 24, 20-28 (2018).
12. Burrell, R. A., McGranahan, N., Bartek, J. & Swanton, C. The causes and consequences of genetic heterogeneity in cancer evolution. Nature 501, 338-345 (2013).
13. Majzner, R. G. & Mackall, C. L. Tumor Antigen Escape from CAR T-cell Therapy. Cancer Discovery 8, 1219 (2018).
14. O'Rourke, D. M. et al. A single dose of peripherally infused EGFRvIII-directed CAR T cells mediates antigen loss and induces adaptive resistance in patients with recurrent glioblastoma. Science Translational Medicine 9, eaaa0984 (2017).
15. Shah, N. N. et al. Bispecific anti-CD20, anti-CD19 CAR T cells for relapsed B cell malignancies: a phase 1 dose escalation and expansion trial. Nature Medicine 26, 1569-1575 (2020).
16. Ruella, M. et al. Dual CD19 and CD123 targeting prevents antigen-loss relapses after CD19-directed immunotherapies. J Clin Invest 126, 3814-3826 (2016).
17. Hegde, M. et al. Tandem CAR T cells targeting HER2 and IL13Rα2 mitigate tumor antigen escape. J Clin Invest 126, 3036-3052 (2016).
18. Choi, B. D. et al. CAR-T cells secreting BiTEs circumvent antigen escape without detectable toxicity. Nature Biotechnology 37, 1049-1058 (2019).
19. Wing, A. et al. Improving CART-Cell Therapy of Solid Tumors with Oncolytic Virus-Driven Production of a Bispecific T-cell Engager. Cancer Immunology Research 6, 605 (2018).
20. Went, P. T. H. et al. Frequent EpCam protein expression in human carcinomas. Human Pathology 35, 122-128 (2004).
21. Dustin, M. L., Rothlein, R., Bhan, A. K., Dinarello, C. A. & Springer, T. A. Induction by IL 1 and interferon-gamma: tissue distribution, biochemistry, and function of a natural adherence molecule (ICAM-1). J Immunol 137, 245-254 (1986).
22. Park, S. et al. Tumor suppression via paclitaxel-loaded drug carriers that target inflammation marker upregulated in tumor vasculature and macrophages. Biomaterials 34, 598-605 (2013).
23. Lisak, R. P. & Bealmear, B. Upregulation of intercellular adhesion molecule-1 (ICAM-1) on rat Schwann cells in vitro: comparison of interferon-gamma, tumor necrosis factor-alpha and interleukin-1. J Peripher Nerv Syst 2, 233-243 (1997).
24. Jahnke, A. & Johnson, J. P. Intercellular Adhesion Molecule 1 (ICAM-1) is Synergistically Activated by TNF-α and IFN-γ Responsive Sites. Immunobiology 193, 305-314 (1995).
25. Trzpis, M., McLaughlin, P. M. J., de Leij, L. M. F. H. & Harmsen, M. C. Epithelial cell adhesion molecule: more than a carcinoma marker and adhesion molecule. Am J Pathol 171, 386-395 (2007).
26. Morgan, R. A. et al. Case report of a serious adverse event following the administration of T cells transduced with a chimeric antigen receptor recognizing ERBB2. Mol Ther 18, 843-851 (2010).
27. Lamers, C. H. J. et al. Treatment of Metastatic Renal Cell Carcinoma With CAIX CAR-engineered T cells: Clinical Evaluation and Management of On-target Toxicity. Molecular Therapy 21, 904-912 (2013).
28. Thistlethwaite, F. C. et al. The clinical efficacy of first-generation carcinoembryonic antigen (CEACAM5)-specific CAR T cells is limited by poor persistence and transient pre-conditioning-dependent respiratory toxicity. Cancer Immunol Immunother 66, 1425-1436 (2017).
29. Goff, S. L. et al. Pilot Trial of Adoptive Transfer of Chimeric Antigen Receptor-transduced T Cells Targeting EGFRvIII in Patients With Glioblastoma. J Immunother 42, 126-135 (2019).
30. Park, S. et al. Micromolar affinity CAR T cells to ICAM-1 achieves rapid tumor elimination while avoiding systemic toxicity. Scientific Reports 7, 14366 (2017).
31. Björk, P. et al. Isolation, partial characterization, and molecular cloning of a human colon adenocarcinoma cell-surface glycoprotein recognized by the C215 mouse monoclonal antibody. J Biol Chem 268, 24232-24241 (1993).
32. Huls, G. A. et al. A recombinant, fully human monoclonal antibody with antitumor activity constructed from phage-displayed antibody fragments. Nature Biotechnology 17, 276-281 (1999).
33. Dohlsten, M. et al. Monoclonal antibody-superantigen fusion proteins: tumor-specific agents for T-cell-based tumor therapy. Proc NatlAcad Sci USA 91, 8945-8949 (1994).
34. Ku, J.-L. & Park, J.-G. Biology of SNU cell lines. Cancer Res Treat 37, 1-19 (2005).
35. Jung, M. et al. Chimeric Antigen Receptor T Cell Therapy Targeting ICAM-1 in Gastric Cancer. Molecular Therapy—Oncolytics 18, 587-601 (2020).
36. Nam, H. J. et al. Evaluation of the antitumor effects and mechanisms of PF00299804, a pan-HER inhibitor, alone or in combination with chemotherapy or targeted agents in gastric cancer. Mol Cancer Ther 11, 439-451 (2012).
37. Motoyama, T., Hojo, H. & Watanabe, H. Comparison of seven cell lines derived from human gastric carcinomas. Acta Pathol Jpn 36, 65-83 (1986).
38. Vedvyas, Y. et al. Longitudinal PET imaging demonstrates biphasic CAR T cell responses in survivors. JCI Insight 1 (2016).
39. Dunn, G. P., Old, L. J. & Schreiber, R. D. The Three Es of Cancer Immunoediting. Annual Review of Immunology 22, 329-360 (2004).
40. Shah, N. N. & Fry, T. J. Mechanisms of resistance to CAR T cell therapy. Nature Reviews Clinical Oncology 16, 372-385 (2019).
41. Rafiq, S., Hackett, C. S. & Brentjens, R. J. Engineering strategies to overcome the current roadblocks in CAR T cell therapy. Nature Reviews Clinical Oncology 17, 147-167 (2020).
42. Min, I. M. et al. CAR T Therapy Targeting ICAM-1 Eliminates Advanced Human Thyroid Tumors. Clinical Cancer Research 23, 7569 (2017).
43. Jung, W.-C. et al. Expression of intercellular adhesion molecule-1 and e-selectin in gastric cancer and their clinical significance. J Gastric Cancer 12, 140-148 (2012).
44. O'Hanlon, D. M. et al. Soluble adhesion molecules (E-selectin, ICAM-1 and VCAM-1) in breast carcinoma. European Journal of Cancer 38, 2252-2257 (2002).
45. Rosette, C. et al. Role of ICAM1 in invasion of human breast cancer cells. Carcinogenesis 26, 943-950 (2005).
46. Sun, J.-J. et al. Invasion and metastasis of liver cancer: expression of intercellular adhesion molecule 1. Journal of Cancer Research and Clinical Oncology 125, 28-34 (1999).
47. Jacoby, E. et al. CD19 CAR immune pressure induces B-precursor acute lymphoblastic leukaemia lineage switch exposing inherent leukaemic plasticity. Nature Communications 7, 12320 (2016).
48. Xia, A., Zhang, Y., Xu, J., Yin, T. & Lu, X.-J. T Cell Dysfunction in Cancer Immunity and Immunotherapy. Front Immunol 10, 1719-1719 (2019).
49. Moon, E. K. et al. Multifactorial T-cell hypofunction that is reversible can limit the efficacy of chimeric antigen receptor-transduced human T cells in solid tumors. Clin Cancer Res 20, 4262-4273 (2014).
50. Chong, E. A. et al. Phase I/II Study of Pembrolizumab for Progressive Diffuse Large B Cell Lymphoma after Anti-CD19 Directed Chimeric Antigen Receptor Modified T Cell Therapy. Blood 130, 4121-4121 (2017).
51. Gray, K. D. et al. PD1 blockade enhances ICAM1-directed CAR T therapeutic efficacy in advanced thyroid cancer. Clinical Cancer Research, clincanres.1523.2020 (2020).
52. Cherkassky, L. et al. Human CAR T cells with cell-intrinsic PD-1 checkpoint blockade resist tumor-mediated inhibition. J Clin Invest 126, 3130-3144 (2016).
53. Liu, X. et al. A Chimeric Switch-Receptor Targeting PD1 Augments the Efficacy of Second-Generation CAR T Cells in Advanced Solid Tumors. Cancer Research 76, 1578 (2016).
54. Condomines, M. et al. Tumor-Targeted Human T Cells Expressing CD28-Based Chimeric Antigen Receptors Circumvent CTLA-4 Inhibition. PLoS One 10, e0130518 (2015).
55. Tang, N. et al. TGF-β inhibition via CRISPR promotes the long-term efficacy of CAR T cells against solid tumors. JCI Insight 5 (2020).
56. Chinnasamy, D. et al. Local Delivery of Interleukin-12 Using T Cells Targeting VEGF Receptor-2 Eradicates Multiple Vascularized Tumors in Mice. Clinical Cancer Research 18, 1672 (2012).
57. Krenciute, G. et al. Transgenic Expression of IL15 Improves Antiglioma Activity of IL13Rα2-CAR T Cells but Results in Antigen Loss Variants. Cancer Immunology Research 5, 571 (2017).
58. Hu, B. et al. Augmentation of Antitumor Immunity by Human and Mouse CAR T Cells Secreting IL-18. Cell Rep 20, 3025-3033 (2017).
59. Zhong, S. et al. T-cell receptor affinity and avidity defines antitumor response and autoimmunity in T-cell immunotherapy. Proceedings of the National Academy of Sciences 110, 6973 (2013).
60. Schmid, D. A. et al. Evidence for a TCR Affinity Threshold Delimiting Maximal CD8 T Cell Function. The Journal of Immunology 184, 4936 (2010).
61. Ghorashian, S. et al. Enhanced CAR T cell expansion and prolonged persistence in pediatric patients with ALL treated with a low-affinity CD19 CAR. Nature Medicine 25, 1408-1414 (2019).
62. Liu, X. et al. Affinity-Tuned ErbB2 or EGFR Chimeric Antigen Receptor T Cells Exhibit an Increased Therapeutic Index against Tumors in Mice. Cancer Research 75, 3596 (2015).

What is claimed is:

1. A dual chimeric antigen receptor (CAR) comprising:
(a) a single-chain variable fragment (scFv) against EpCAM,
(b) an I domain of the α_{L} subunit of human lymphocyte function-associated antigen-1 that binds to ICAM-1,
(c) at least one transmembrane domain,
(d) at least one co-stimulatory domains, and
(iv) at least one activating domain.

2. The dual CAR of claim 1, comprises an EpCAM CAR targeting EpCAM and an ICAM-1 CAR targeting ICAM-1, wherein the EpCAM CAR comprises scFv against EpCAM, one transmembrane domain, one or more co-stimulatory domains, and one activating domain, and the ICAM-1 CAR comprises an I domain, one transmembrane domain, one or more co-stimulatory domains, and one activating domain.

3. The dual CAR of claim 1, comprises from N-terminus to C-terminus scFv against EpCAM, I domain, a transmembrane domain, one or more co-stimulatory domains, and an activating domain.

4. The dual CAR of claim 1, comprises from N-terminus to C-terminus I domain, scFv against EpCAM, a transmembrane domain, one or more co-stimulatory domains, and an activating domain.

5. The dual CAR of claim 1, wherein the CAR binds to EpCAM with an affinity between about 50 nM and 50 μM.

6. The dual CAR of claim 5, wherein the CAR binds to EpCAM with an affinity between about between 80 nM and 20 μM.

7. The dual CAR of claim 1, wherein the scFv against EpCAM comprises heavy chain variable CDR1 of the amino acid sequence of SEQ ID NO: 9, heavy chain variable CDR2 of the amino acid sequence of SEQ ID NO: 10, and heavy chain variable CDR3 of the amino acid sequence of SEQ ID NO: 2, 3, 4, 5, 6, 7, or 8.

8. The dual CAR of claim 7, wherein the scFv against EpCAM comprises light chain variable CDR1 of the amino acid sequence of SEQ ID NO: 11, light chain variable CDR2 of the amino acid sequence of SEQ ID NO: 12, and light chain variable CDR3 of the amino acid sequence of SEQ ID NO: 13.

9. The dual CAR of claim 1, wherein the CAR binds ICAM-1 with an affinity between about 50 nM and about 50 μM.

10. The dual CAR of claim 9, wherein the I domain comprises the sequence of 130-310 amino acids of SEQ ID NO: 1, with one mutation of F292A, F292S, L289G, F265S, or F292G, wherein the numbering of the amino acid residues corresponds to the amino acid residues of SEQ ID NO: 1.

11. The dual CAR of claim 9, wherein the I domain comprises the sequence of 130-310 amino acids of SEQ ID NO: 1, with two mutations of K287C and K294C, wherein the numbering of the amino acid residues corresponds to the amino acid residues of SEQ ID NO: 1.

12. The dual CAR of claim 1, wherein the co-stimulatory domain is selected from the group consisting of CD28, 4-1BB, ICOS-1, CD27, OX-40, GITR, and DAP10.

13. The dual CAR of claim 1, wherein the activating domain is CD3 zeta.

14. The dual CAR of claim 1, further comprising a reporter molecule Somatostatin receptor type 2 (SSTR2).

15. An isolated nucleic acid sequence encoding the dual CAR of claim 1.

16. T cells or natural killer cells modified to express the dual CAR of claim 1.

17. An adoptive cell therapy method for treating cancer, comprising the steps of: administering the CAR-T cells of claim 16 to a subject suffering from cancer.

18. The method according to claim 17, wherein the cancer is gastric cancer, pancreatic cancer, thyroid cancer, or breast cancer.


SEQUENCE LISTING (22 sequences)

SEQ ID NO: 1 - protein, 1145 aa, Homo sapiens
YNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLYPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 2 - protein, 6 aa, Homo sapiens
DPFLHY

SEQ ID NO: 3 - protein, 6 aa, Homo sapiens
DPFLHA

SEQ ID NO: 4 - protein, 6 aa, Homo sapiens
DPFLHL

SEQ ID NO: 5 - protein, 6 aa, Homo sapiens
DPFLHV

SEQ ID NO: 6 - protein, 6 aa, Homo sapiens
DPFLHF

SEQ ID NO: 7 - protein, 6 aa, Homo sapiens
APFLHY

SEQ ID NO: 8 - protein, 6 aa, Homo sapiens
DPFAHY

SEQ ID NO: 9 - protein, 7 aa, Homo sapiens
GGTFSSY

SEQ ID NO: 10 - protein, 6 aa, Homo sapiens
IPIFGT

SEQ ID NO: 11 - protein, 16 aa, Homo sapiens
RSSQSLLHSNGYNYLD

SEQ ID NO: 12 - protein, 7 aa, Homo sapiens
LGSNRAS

SEQ ID NO: 13 - protein, 8 aa, Homo sapiens
MQALQTFT

SEQ ID NO: 14 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHYWGQGTLVT

SEQ ID NO: 15 - protein, 110 aa, Homo sapiens
EIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEI

SEQ ID NO: 16 - protein, 111 aa, Homo sapiens
EIELTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTFTFGPGTKVEIK

SEQ ID NO: 17 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHAWGQGTLVT

SEQ ID NO: 18 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHLWGQGTLVT

SEQ ID NO: 19 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHVWGQGTLVT

SEQ ID NO: 20 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARAPFLHYWGQGTLVT

SEQ ID NO: 21 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFAHYWGQGTLVT

SEQ ID NO: 22 - protein, 112 aa, Homo sapiens
QVQLVQSGAEVKKPGSSVRVSCKASGGTFSSYAISWVRQAPGQGLEWMGGIIPIFGTANYAQKFQGRVTITADESTSTAYMELSSLRSEDTAVYYCARDPFLHFWGQGTLVT